(19) United States
(12) Patent Application Publication
(10) Pub. No.: US 2025/0201419 A1
(43) Pub. Date: Jun. 19, 2025

(54) METHODS FOR CHARACTERIZING AN ADNEXAL MASS

(71) Applicant: Aspira Women’s Health Inc., Austin, TX (US)

(72) Inventors: Todd C. Pappas, Austin, TX (US); Ryan T. Phan, Austin, TX (US); Nitin Bhardwaj, Austin, TX (US); Daniel R. Ure, Austin, TX (US); Srinka Ghosh, Austin, TX (US)

(21) Appl. No.: 18/898,153

(22) Filed: Sep. 26, 2024

Related U.S. Application Data
(63) Continuation of application No. PCT/US23/16520, filed on Mar. 28, 2023.
(60) Provisional application No. 63/325,046, filed on Mar. 29, 2022.
(60) Provisional application No. 63/484,998, filed on Feb. 14, 2023.

(51) Int. Cl. G16H 50/30; G01N 33/574

(52) U.S. Cl. CPC G16H 50/30; G01N 33/57449; G01N 2333/775; G01N 2800/50

(57) ABSTRACT

The present invention provides methods for the assessment of an adnexal mass predetermined to be benign or asymptomatic (e.g., asymptomatic or benign adnexal mass) in a variety of subjects (e.g., pre- and post-menopausal women). In particular, the present invention provides methods for determining the malignancy risk of ovarian tumors in selected subjects (e.g., benign or indeterminate risk).

### CROSS-REFERENCE TO RELATED APPLICATIONS

This application is a continuation of International Application No. PCT/US2023/016520, filed Mar. 28, 2023, which claims priority to and the benefit of U.S. Provisional Application Nos. 63/325,046, filed Mar. 29, 2022, and 63/484,998, filed Feb. 14, 2023, respectively, the disclosures of each of which is incorporated herein by reference in its entirety.

### BACKGROUND OF THE INVENTION

Adnexal masses are a common gynecological condition. With approximately 10% of women undergoing surgery for an adnexal mass during their lifetime, the research efforts to date have focused on tools designed to identify which of these masses are cancerous. Ovarian cancer is the deadliest gynecological cancer, therefore prompt and correct identification of malignancies is crucial. However, the incidence of ovarian cancer is still relatively low. Approximately 85% of masses in premenopausal women will be benign, so testing that can accurately differentiate malignant masses from those that require less extensive intervention and treatment is of clinical value.

### SUMMARY OF THE INVENTION

As described below, the present invention features methods of assessing ovarian cancer risk in a subject (e.g., a subject having an adnexal mass previously determined to be non-malignant or asymptomatic) using a panel of biomarkers.

In one aspect, the present disclosure features a method for assessing a selected subject's risk of having ovarian cancer. The method includes selecting a subject having an adnexal mass previously characterized as non-malignant or asymptomatic. The method further includes characterizing a panel of markers in a biological sample derived from the selected subject to determine a score, where the markers in the panel of markers include cancer antigen 125 (CA125), human epididymis protein 4 (HE4), beta-2 microglobulin (B2M), apolipoprotein A-1 (ApoA1), transferrin, transthyretin, and follicle stimulating hormone (FSH), and where the score identifies the subject as having a benign adnexal mass or an adnexal mass with an indeterminate risk of malignancy.

In another aspect, the present disclosure features a method of conservative management of an adnexal mass in a selected subject. The method includes selecting a subject having an adnexal mass and at least one contraindication to surgical intervention. The method further includes characterizing a panel of markers in a biological sample derived from the selected subject to determine a score, where the markers in the panel of markers include cancer antigen 125 (CA125), human epididymis protein 4 (HE4), beta-2 microglobulin (B2M), apolipoprotein A-1 (ApoA1), transferrin, transthyretin, and follicle stimulating hormone (FSH), and where the score identifies the subject as having a benign adnexal mass, or having an adnexal mass having an indeterminate risk of malignancy. The method further includes conservatively managing the adnexal mass when the score identifies the subject as having a benign adnexal mass.

In another aspect, the present disclosure features a computer implemented method for assessing a subject's risk of having ovarian cancer. The method includes receiving, by one or more computing devices each comprising a processor and a memory, a plurality of signals, each signal representing a value of a biomarker from a panel of biomarkers detected in a biological sample derived from a subject having an adnexal mass, where the panel of biomarkers includes Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH).

The method further includes receiving, by the one or more computing devices, an age value representing the age of the subject and a menopausal value representing the menopausal state of the subject. The method further includes determining, using an artificial neural network stored in the one or more computing devices, a score based on the plurality of signals, the age value, and the menopausal value, where the score represents whether the adnexal mass is benign, or the adnexal mass has an indeterminate risk of malignancy.

In another aspect, the present disclosure features a method for training an artificial neural network for detecting the risk of ovarian cancer in a subject. The method includes collecting a training set comprising a set of malignant adnexal mass samples and a set of benign adnexal mass samples. The method further includes balancing the number of samples in each of the set of malignant adnexal mass samples and the set of benign adnexal mass samples by synthetically creating samples near the decision boundary. The method further includes training the artificial neural network on the training set, wherein the training comprises regularizing the artificial neural network using node dropout and attaching a higher weight to identifying malignant samples.

In another aspect, the present disclosure features a method for monitoring a subject's risk of having ovarian cancer. The method includes: (a) assessing the subject at a first time point in a plurality of time points using any of the methods, aspects, or embodiments described herein. The method further includes: (b) repeating step (a) in one or more biological samples from the subject identified as having an intermediate or low ovarian cancer risk, or as having a benign adnexal mass, at one or more following time points in the plurality of time points, thereby monitoring the subject.

In any of the above aspects, or embodiments thereof, the contraindication is a comorbidity precluding surgical intervention, a risk of harming fertility in the subject, size of the adnexal mass, lack of pain in the adnexal mass.

In any of the above aspects, or embodiments thereof, the conservative management includes delaying or avoiding surgical intervention in the selected subject.

In any of the above aspects, or embodiments thereof, the plurality of signals each represent a biomarker spectrum peak detected for each biomarker of the panel of biomarkers.

In any of the above aspects, or embodiments thereof, the artificial neural network is a deep feed-forward neural network.

In any of the above aspects, or embodiments thereof, the artificial neural network includes a plurality of input nodes, a plurality of hidden nodes, and a plurality of output nodes. In any of the above aspects, or embodiments thereof, each of the input nodes includes a memory location for storing an input value. In any of the above aspects, or embodiments thereof, each input value corresponds to a different value from one of the plurality of signals, the age value, or the menopausal value. In any of the above aspects, or embodiments thereof, the plurality of hidden nodes is organized into a plurality of hidden layers, each hidden layer having a different set of weighted nodes and/or activation functions. In any of the above aspects, or embodiments thereof, the plurality of output nodes includes a first output node and a second output node, the first output node including a memory location for storing a first output value indicating the probability of a first classification, and the second output node including a memory location for storing a second output value indicating the probability of a second classification. In any of the above aspects, or embodiments thereof, the first classification represents a benign adnexal mass and the second classification represents an adnexal mass having an indeterminate risk of malignancy.

In any of the above aspects, or embodiments thereof, the artificial neural network uses the softmax function to assign the first and second output values.

In any of the above aspects, or embodiments thereof, the artificial neural network is regularized using node dropout to reduce overfitting.

In any of the above aspects, or embodiments thereof, the artificial neural network is trained using supervised training. In any of the above aspects, or embodiments thereof, the artificial neural network is trained using a training set comprising a set of malignant samples and a set of benign samples. In any of the above aspects, or embodiments thereof, the number of samples in the set of malignant samples and the number of samples in the set of benign samples is balanced using a synthetic minority oversampling technique (SMOTE) to create a balanced training set. In any of the above aspects, or embodiments thereof, the SMOTE includes balancing minority and majority classes within the training set by creating synthetic samples near the decision boundary. In any of the above aspects, or embodiments thereof, the balanced training set has an equal amount of malignant samples and benign samples. In any of the above aspects, or embodiments thereof, the training set has 100-500 malignant samples in the set of malignant samples. In any of the above aspects, or embodiments thereof, the artificial neural network is trained by attaching a higher weight to detection of malignant samples.

In any of the above aspects, or embodiments thereof, the subject has an adnexal mass previously characterized as non-malignant or asymptomatic. In any of the above aspects, or embodiments thereof, the characterization of the adnexal mass as non-malignant or asymptomatic comprises using one or more of imaging or biomarker screening. In any of the above aspects, or embodiments thereof, the imaging is transvaginal ultrasonography (TVUS). In any of the above aspects, or embodiments thereof, the characterization of the adnexal mass as non-malignant or asymptomatic includes using TVUS imaging over the course of at least 5 months without an increase in adnexal mass size. In any of the above aspects, or embodiments thereof, the biomarker screening is CA125 or HE4 screening.

In any of the above aspects, or embodiments thereof, the characterizing includes any of the computer implemented methods, aspects, or embodiments described herein.

In any of the above aspects, or embodiments thereof, the biological sample is a serum sample.

In any of the above aspects, or embodiments thereof, the training set is derived from immunoassays.

In any of the above aspects, or embodiments thereof, the one or more time points are separated by 3-6 months. In any of the above aspects, or embodiments thereof, the one or more time points are separated by 3 months.

In any of the above aspects, or embodiments thereof, the subject is recommended for clinical follow-up when a score change of greater than 2.25 between two successive time points in the plurality of time points is detected.

In any of the above aspects, or embodiments thereof, when the score represents the adnexal mass as having an indeterminate risk of malignancy the score further represents whether the adnexal mass has an intermediate risk of malignancy or a high risk of malignancy.

In any of the above aspects, or embodiments thereof, the score is normalized to a 10 point scale. In any of the above aspects, or embodiments thereof, a score of less than 2.5 represents a benign adnexal mass, a score of 2.5 to 5 represents an adnexal mass having an intermediate risk of malignancy, and a score of greater than 5 represents an adnexal mass having a high risk of malignancy.

As described in detail herein, any method known in the art can be used to measure a panel of biomarkers. In aspects of the invention, the panel of biomarkers are measured using any immunoassay well known in the art. In embodiments, the immunoassay can be, but is not limited to, ELISA, western blotting, and radioimmunoassay.

Compositions and articles defined by the invention were isolated or otherwise manufactured in connection with the examples provided below. Other features and advantages of the invention will be apparent from the detailed description, and from the claims

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia ofMolecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “adnexal mass” is meant an abnormal growth that develops near the uterus, most commonly arising from the ovaries, fallopian tubes, or connective tissues. The lump-like mass can be cystic (fluid-filled) or solid. Adnexal masses may be benign (non-cancerous) or malignant (cancerous). Adnexal masses may be symptomatic or asymptomatic. By a “symptomatic adnexal mass” is meant an adnexal mass that presents symptoms in a patient. The symptoms may include, but are not limited to, abdominal fullness, abdominal bloating, pelvic pain, difficulty with bowel movements, and increased frequency of urination, abnormal vaginal bleeding, or pelvic pressure. By “asymptomatic adnexal mass” is meant an adnexal mass producing or showing no symptoms in a patient. By “non-malignant adnexal mass” is meant an adnexal mass determined by a medical professional to be non-malignant.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard art known methods such as those described herein. An alteration may be by as little as 1%, 2%, 3%, 4%, 5%, 10%, 20%, 30%, or by 40%, 50%, 60%, or even by as much as 70%, 75%, 80%, 90%, or 100%.

By “benign” is meant a condition or growth (e.g., an adnexal mass) that is non-cancerous.

By “biologic sample” is meant any tissue, cell, fluid, or other material derived from an organism. In embodiments, the sample is a serum, blood, or plasma sample. In other embodiments, the sample is a biopsy sample obtained from a subject having an abnormal growth (e.g., an adnexal mass).

A “biomarker” or “marker” as used herein generally refers to a protein, nucleic acid molecule, clinical indicator, or other analyte that is associated with a disease. In one embodiment, a marker of ovarian cancer is differentially present in a biological sample obtained from a subject having or at risk of developing ovarian cancer relative to a reference. A marker is differentially present if the mean or median level of the biomarker present in the sample is statistically different from the level present in a reference. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing ovarian cancer, for determining the prognosis of the subject, for evaluating therapeutic efficacy, or for selecting a treatment regimen (e.g., selecting that the subject be evaluated and/or treated by a surgeon that specializes in gynecologic oncology).

Markers useful in the panels of the invention include, for example, FSH, HE4, CA125, transthyretin, transferrin, ApoA1, and 02 microglobulin proteins, as well as the nucleic acid molecules encoding such proteins. Fragments useful in the methods of the invention are sufficient to bind an antibody that specifically recognizes the protein from which the fragment is derived. The invention includes markers that are substantially identical to the following sequences. Preferably, such a sequence is at least 85%, 90%, 95% or even 99% identical at the amino acid level or nucleic acid to the sequence used for comparison.

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “Follicle-stimulating hormone (FSH) polypeptide” is meant a polypeptide or fragment thereof having at least about 85% amino acid identity to NCBI Accession No. NP_000501.

By “Human Epididymis Protein 4 (HE4) polypeptide” is meant a polypeptide or fragment thereof having at least about 85% amino acid identity to NCBI Accession No. NP_006094.

By “Cancer Antigen 125 (CA125) polypeptide” is meant a polypeptide or fragment thereof having at least about 85% amino acid identity to Swiss-Prot Accession number Q8WXI7.

By “Transthyretin (Prealbumin) polypeptide” is meant a polypeptide or fragment thereof having at least about 85% amino acid identity to Swiss Prot Accession number P02766.

By “Transferrin polypeptide” is meant a polypeptide or fragment thereof having at least about 85% amino acid identity to UniProtKB/TrEMBL Accession number Q06AH7.

By “Apolipoprotein A1 (ApoA1) polypeptide” is meant a polypeptide or fragment thereof having at least about 85% amino acid identity to Swiss Prot Accession number P02647.

By “3-2 microglobulin polypeptide” is meant a polypeptide or fragment thereof having at least about 85% amino acid identity to SwissProt Accession No. P61769.

Select exemplary sequences delineated herein are shown in FIG. 1A.

By “capture reagent” is meant a reagent that specifically binds a nucleic acid molecule or polypeptide to select or isolate the nucleic acid molecule or polypeptide.

By “clinical aggressiveness” is meant the severity of the neoplasia. Aggressive neoplasias are more likely to metastasize than less aggressive neoplasias. While conservative methods of treatment are appropriate for less aggressive neoplasias, more aggressive neoplasias require more aggressive therapeutic regimens.

By “decision boundary” is meant the separation or distinction between classes in a classification system. When the decision boundary is crossed, a classification system, such as an artificial neural network, will change classifications from one class to another class.

By “deep feed-forward neural network” is meant a neural network in which the connections between artificial neurons or nodes do not form loops or cycles. In some embodiments, these neural networks feature a layer of input nodes, a layer of output nodes, and one or more layers of hidden nodes situated between the input nodes and output nodes. In preferred embodiments, information is only passed forward between these layers, for example, from the input layer, to the one or more hidden layers, to the output layer. By “input nodes” is meant one or more nodes or artificial neurons which are configured to receive input. By “output nodes” is meant one or more nodes or artificial neurons which are configured to provide an output for the neural network (for example, such as classifications, or predicted probabilities of forecasted features). In exemplary embodiments, the output nodes provide a classification which indicates the probability of a low risk of malignancy or an elevated risk of malignancy for an adnexal mass. By “hidden nodes” is meant one or more nodes or artificial neurons which are not input nodes or output nodes, which each transform input provided to such nodes before passing the transformed information on to another node. In some embodiments, the transformation of provided input by hidden nodes may include applying a weight and/or a bias. Weights, or weighted nodes, may, in some embodiments, represent the strength, magnitude, and/or importance of connections between nodes. In some embodiments, weights and biases may change during training. In some embodiments, the transformation of provided input by hidden nodes may include an activation function, which defines the output of a given hidden node based on the input provided to the node. In some embodiments, the activation function may be linear or non-linear. Activation functions may include ridge functions, radial functions, and fold functions. Exemplary, but non limiting, examples of activation functions include: identity; binary step; logistic, sigmoid, or soft step; hyperbolic tangent (tanh); rectified linear unit (ReLU); Gaussian error linear unit (GELU); Softplus; Exponential linear unit (ELU); scaled exponential linear unit (SELU); leaky rectified linear unit (Leaky ReLU); parametric rectified linear unit (PReLU); signmoid linear unit (SiLU, Sigmoid shrinkage, SiL, or Swish-1); Gaussian; Softmax; Maxout.

As used herein, the terms “determining,” “assessing,” “assaying,” “measuring” and “detecting” refer to both quantitative and qualitative determinations of an analyte, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include ovarian cancer. Examples of conditions include an adnexal mass.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “indeterminate risk of malignancy” is meant a an uncertain, or highly uncertain risk of malignancy (e.g., in an adnexal mass).

By “isolated biomarker” or “purified biomarker” is meant at least 60%, by weight, free from proteins and naturally-occurring organic molecules with which the marker is naturally associated. Preferably, the preparation is at least 75%, more preferably 80, 85, 90 or 95% pure or at least 99%, by weight, a purified isolated biomarker.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “marker profile” is meant a characterization of the expression or expression level of two or more polypeptides or polynucleotides.

By “neoplasia” is meant any disease that is caused by or results in inappropriately high levels of cell division, inappropriately low levels of apoptosis, or both. Examples of cancers include, without limitation, ovarian cancer By “node dropout” is meant the random omission of nodes of the artificial neural network duing training. In some embodiments, node dropout is an effective method for reducing or preventing overfitting.

As used herein, “obtaining” as in “obtaining an agent” includes synthesizing, purchasing, or otherwise acquiring the agent.

The term “ovarian cancer” refers to both primary ovarian tumors as well as metastases of the primary ovarian tumors that may have settled anywhere in the body.

The term “ovarian cancer status” refers to the status of the disease in the patient. Examples of types of ovarian cancer statuses include, but are not limited to, the subject's risk of cancer, the presence or absence of disease, the stage of disease in a patient, and the effectiveness of treatment of disease. In embodiments, a subject identified as having a pelvic mass is assessed to identify if their ovarian cancer status is benign or malignant.

By “overfitting” is meant the production of an analysis which corresponds too closely or exactly to a particular set of data, and may therefore fail to fit additional data or predict future observations reliably. In some embodiments, overfitting results in accurate predictions by the artificial neural network when inputting the training data, but inaccurate predictions when new data is provided. In some embodiments, overfitting may result in poor performance on validation sets after training.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “reduces” is meant a negative alteration. In some embodiments, the alteration is reduced by at least 5%, 10%, 25%, 50%, 75%, or 100%.

By “reference” is meant a standard or control condition of comparison. For example, the marker level(s) present in a patient sample may be compared to the level of the marker in a corresponding healthy cell or tissue or in a diseased cell or tissue (e.g., a cell or tissue derived from a subject having ovarian cancer).

By “sample” is meant a biologic sample such as any tissue, cell, fluid, or other material derived from an organism.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “specifically binds” is meant a compound (e.g., antibody) that recognizes and binds a molecule (e.g., polypeptide), but which does not substantially recognize and bind other molecules in a sample, for example, a biological sample.

By “spectrum peak” is meant a peak of a detection signal for a given biomarker, produced by any appropriate assay, such as, but not limited to, a photometric assay. In some embodiments, commercial modules designed for low, medium, or high throughput assay of biomarkers, may be used to produce such detection signals and/or spectrum peaks, such as, but not limited to, the cobas® 6000 analyzer series (Roche Diagnostics Corporation, Indianapolis, IN, USA).

The accuracy of a diagnostic test can be characterized using any method well known in the art, including, but not limited to, a Receiver Operating Characteristic curve (“ROC curve”). An ROC curve shows the relationship between sensitivity and specificity. Sensitivity is the percentage of true positives that are predicted by a test to be positive, while specificity is the percentage of true negatives that are predicted by a test to be negative. An ROC is a plot of the true positive rate against the false positive rate for the different possible cutpoints of a diagnostic test. Thus, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test. In embodiments, biomarkers and diagnostic methods of the present invention have an AUC greater than 0.50, greater than 0.60, greater than 0.70, greater than 0.80, or greater than 0.90.

Other useful measures of the utility of a test are positive predictive value (“PPV”) and negative predictive value (“NPV”). PPV is the percentage of actual positives who test as positive. NPV is the percentage of actual negatives that test as negative.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

Any compositions or methods provided herein can be combined with one or more of any of the other compositions and methods provided herein.

### BRIEF DESCRIPTION OF THE DRAWINGS

FIG. 1A provides the sequences of select biomarker polypeptides.

FIG. 1B provides a flowchart illustrating the workflow of the development and validation of the algorithm. B and M indicate benign and malignant samples, respectively.

FIG. 2 provides a flowchart illustrating the workflow of an analytical validation exercise exemplified in Example 1 of this disclosure.

FIG. 3 provides graphs showing receiver operating characteristics (ROC) and Precision-Recall curves for the algorithm. In these graphs, area under receiver operating characteristics curve (AUROC) is 0.938, and area under precision recall curve (AUPRC) is 0.700.

FIG. 4 provides a correlation matrix showing correlations between the features used in the algorithm exemplified in Example 1 of this disclosure.

FIG. 5 provides a graph illustrating a variable importance analysis of the features used in the algorithm exemplified in Example 1 of this disclosure.

FIG. 6 provides a flowchart illustrating a workflow diagram showing an analysis data set stratified by physician assessment of malignancy risk. A comprehensive retrospective validation was performed on 2,000 samples with 98 malignant and 1,902 benign specimens. Within these data, 1,640 received an independent physician clinical assessment—using imaging and other clinical examination—as either benign or malignant. A total of 1453 patients were independently assessed as benign by physician, prior to surgery.

FIG. 7 provides a graph showing characteristics of MIA3G in the retrospective validation set. FIG. 7 provides a principal components analysis visualization bi-plot visualizing of the coordinates of biomarker and clinical variables used in derivation of MIA3G, and the individual subjects plotted on the first two principal component dimensions. The data set is the original 2000 patients from the previously published validation. Abbreviates for biomarkers are in the Methods section, Meno=Menopausal status and is a binary variable (pre- or post-menopausal).

FIG. 8 provides a graph illustrating performance of MIA3G in the Multivariate Index Assay Benign (MIAB) data set. FIG. 8 provides a receiver-Operator Characteristics (ROC) plot of the MIAB data set. The area under the ROC curve (AUC) for MIA3G was 0.911.

FIG. 9 provides a graph illustrating the probability of malignancy as a function of MIA3G in the MIAB dataset.

FIG. 10 provides a flowchart illustrating a work-flow diagram showing stratification of samples from prospective studies into the Prospective “Real World” (PRW) and Independent High-Prevalence (IHP) data sets.

FIG. 11 provides a flowchart and a chart illustrating performance of MIA3G in the PRW study. FIG. 11, top, provides a flow diagram showing patient data represented. FIG. 11, bottom, shows the performance for all patients, only those who went to surgery and sensitivity for epithelial ovarian cancer (EOC) malignancies.

FIG. 12 provides charts showing predicted performance of MIA3G derived from the PRW dataset. FIG. 12, left, provides negative predictive value (NPV) plotted as a function of MIA3G cut-off score. Individual lines represent NPV over MIA3G score cut-off by predicted prevalences from 1.25-10%. Note the y-axis break to emphasize the effects of prevalence on NPV. FIG. 12, right, provides a logistic regression of the probability of malignancy as a function of MIA3G score.

FIG. 13 provides charts showing the results of bootstrap analysis to evaluate the effects of prevalence on MIA3G performance estimates and variability. Each statistic was estimated over 5000 bootstrap samples at prevalence of 1-10%. The line represents the median estimated statistic, the gray band is the 2.5-97.5 percentile of the distributions. Bootstrap estimates are shown for sensitivity (FIG. 13, top left), specificity (FIG. 13, top middle), Accuracy (FIG. 13, top right), positive predictive value (PPV) (FIG. 13, bottom left) and NPV (FIG. 13, bottom right). Note y-axis breaks on FIG. 13, top middle and bottom right, to emphasize prevalence dependent changes.

### DETAILED DESCRIPTION OF THE INVENTION

The disclosure provides for the use of a panel of biomarkers for characterizing an adnexal (e.g., as non-malignant or asymptomatic).

The invention is based, at least in part, on the discovery of a deep feed-forward neural network for ovarian cancer risk assessment, using 7 protein biomarkers along with age and menopausal status as input features. The algorithm was developed on a heterogenous dataset of 1,067 serum specimens from women with adnexal masses (prevalence=31.8%). It was subsequently validated on a cohort almost twice that size (N=2,000). In the analytical validation dataset (prevalence=4.9%), MIA3G demonstrated a sensitivity of 89.8% and a specificity of 84.02%. The positive predictive value was 22.45%, and the negative predictive value was 99.38%. When stratified by cancer type and stage, MIA3G achieved a sensitivity of 94.94% for epithelial ovarian cancer, 76.92% for early-stage and 98.04% for late-stage cancer. The balanced performance of MIA3G leads to a high sensitivity and high specificity, a combination which may be clinically useful for providers in evaluating the appropriate management strategy for their patients. Limitations of this work include the largely retrospective nature of the dataset as well as the unequal, albeit random, assignment of histologic subtypes between the training and validation data sets. Future directions may include the addition of new biomarkers or other modalities to strengthen the performance of the algorithm.

### Adnexal Masses

Adnexal masses are a common gynecological condition. With approximately 10% of women undergoing surgery for an adnexal mass during their lifetime, the research efforts to date have focused on tools designed to identify which of these masses are cancerous. Ovarian cancer is the deadliest gynecological cancer, therefore prompt and correct identification of malignancies is crucial. However, the incidence of ovarian cancer is still relatively low. Approximately 85% of masses in premenopausal women will be benign, so testing that can accurately differentiate malignant masses from those that require less extensive intervention and treatment is of clinical value. The vast majority of women have adnexal masses that can be managed conservatively, for example, by watchful waiting. Such conservative approaches can advantageously preserve fertility without subject women to unnecessary surgical intervention.

### Biomarkers

In particular embodiments, a biomarker is an organic biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease.

### Biomarkers for Ovarian Cancer

The invention provides a panel of polypeptide or polynucleotide biomarkers that are differentially present in subjects having ovarian cancer, in particular, a benign vs. malignant pelvic mass. The biomarkers of this invention are differentially present depending on ovarian cancer status, including subjects having ovarian cancer vs. subjects that do not have ovarian cancer, or menopausal status, including subjects that are pre- or post-menopausal.

The biomarker panel of the invention comprises one or more of the biomarkers presented in the following Table 1.

TABLE 1

| | Differential |
|---|---|
| | Regulation in |
| Biomarker | ovarian cancer |
| ApoA1 | Decreased |
| Beta2 Microglobulin | Increased |
| (B2M) | |
| Insulin-like growth | Increased |
| factor binding protein | |
| (IGFBP2) | |
| Follicle-stimulating | Increased |
| hormone (FSH) | |
| Human Epididymis | Increased |
| Protein 4 (HE4 | |
| Cancer Antigen 125 | Increased |
| (CA125) | |
| Transthyretin | Decreased |
| (prealbumin) | |
| Transferrin | Decreased |

As would be understood, references herein to a biomarker of Table 1, a panel of biomarkers, or other similar phrase indicates one or more of the biomarkers set forth in Table 1 or otherwise described herein. A panel of one or more of the biomarkers of Table 1 may be used in combination with one or more panels of one or more of the biomarkers of Table 1. For example, in one embodiment, a panel comprising biomarkers ApoA1, CA125, 02M, Transferrin, TT, FSH, and HE4 may be used in combination with a panel comprising ApoA1, CA125, 02M, Transferrin, and TT. In one embodiment, a panel comprising biomarkers ApoA1, CA125, 02M, Transferrin, TT, FSH, and HE4 may be used in combination with a panel comprising Follicle-stimulating hormone FSH, CA125, HE4, ApoA1, and Transferrin. In one embodiment, a panel comprising biomarkers ApoA1, CA125, 02M, Transferrin, TT, FSH, and HE4 may be used in combination with a panel comprising ApoA1, CA125, 02M, Transferrin, and TT and a panel comprising Follicle-stimulating hormone FSH, CA125, HE4, ApoA1, and Transferrin.

The invention provides panels comprising isolated biomarkers. The biomarkers can be isolated from biological fluids, such as urine or serum. They can be isolated by any method known in the art. In certain embodiments, this isolation is accomplished using the mass and/or binding characteristics of the markers. For example, a sample comprising the biomolecules can be subject to chromatographic fractionation and subject to further separation by, e.g., acrylamide gel electrophoresis. Knowledge of the identity of the biomarker also allows their isolation by immunoaffinity chromatography. By “isolated biomarker” is meant at least 60%, by weight, free from proteins and naturally-occurring organic molecules with which the marker is naturally associated. Preferably, the preparation is at least 75%, more preferably 80, 85, 90 or 95% pure or at least 99%, by weight, a purified isolated biomarker.

### Follicle-Stimulating Hormone (FSH)

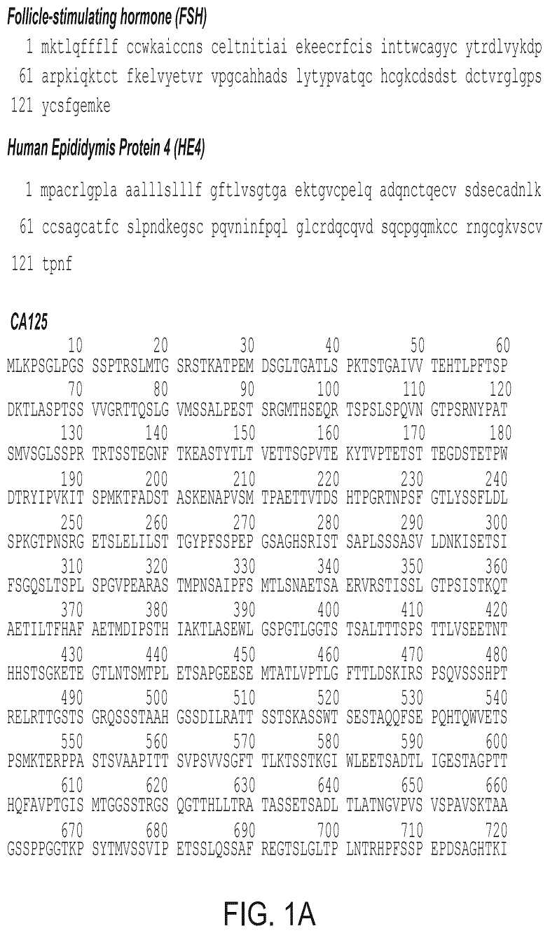

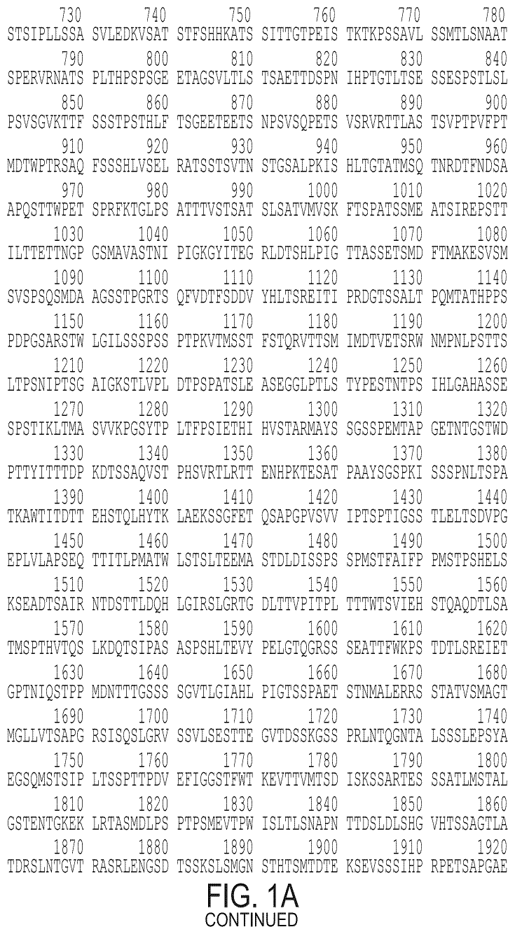

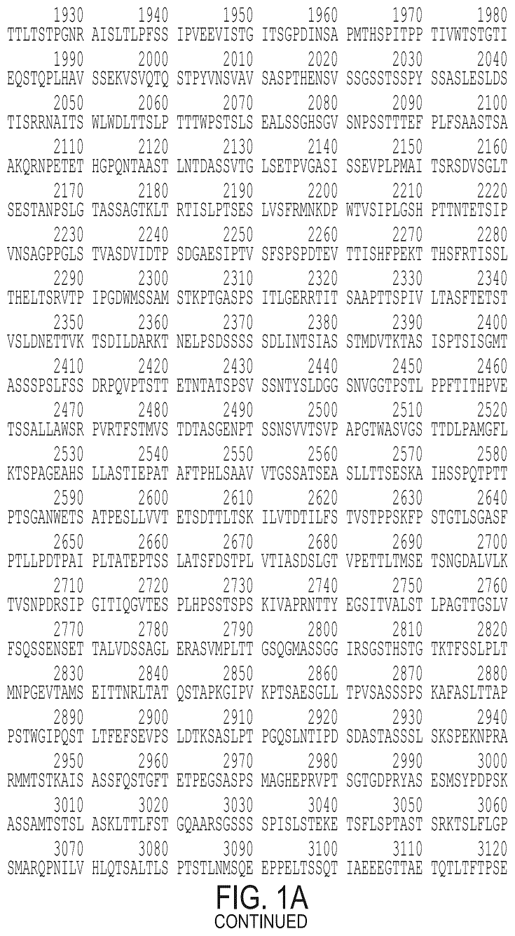

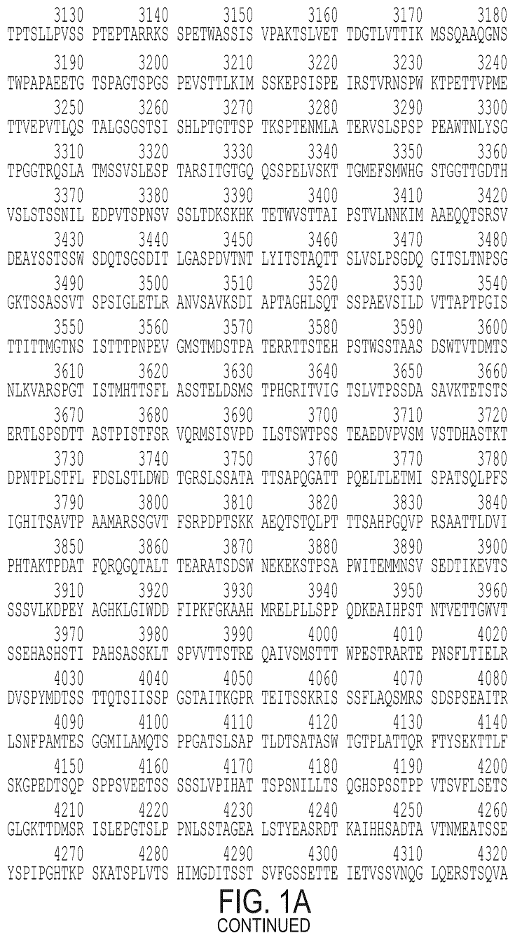

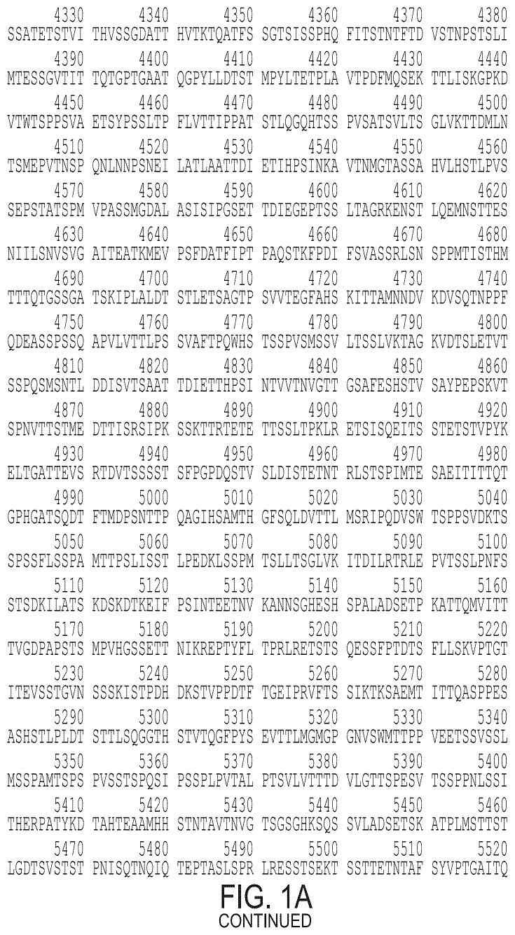

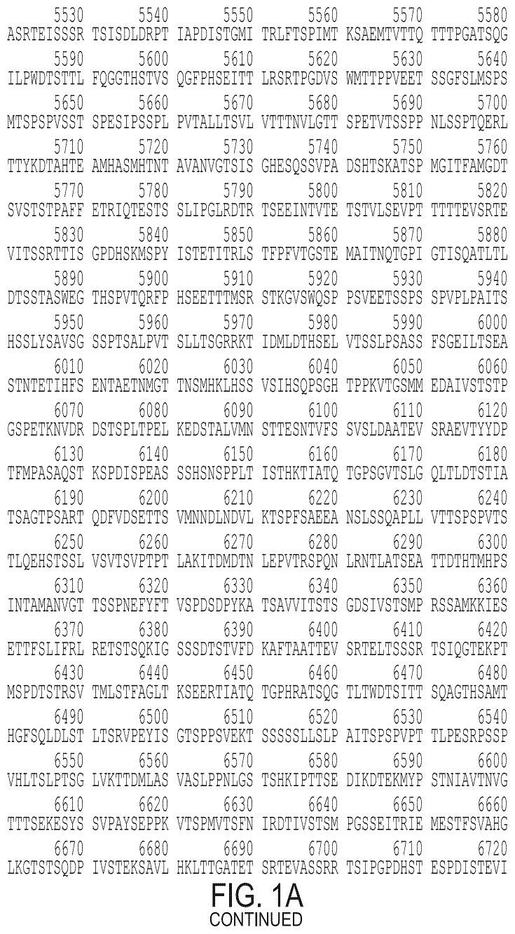

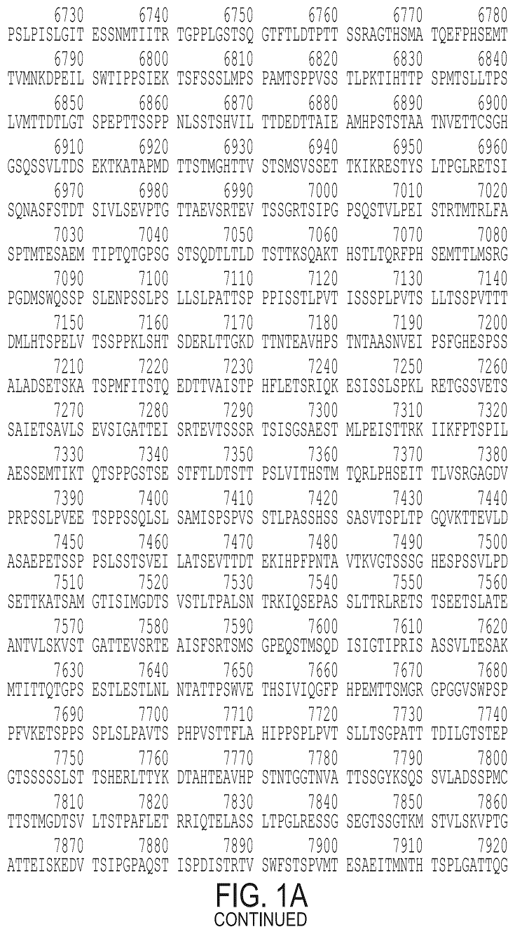

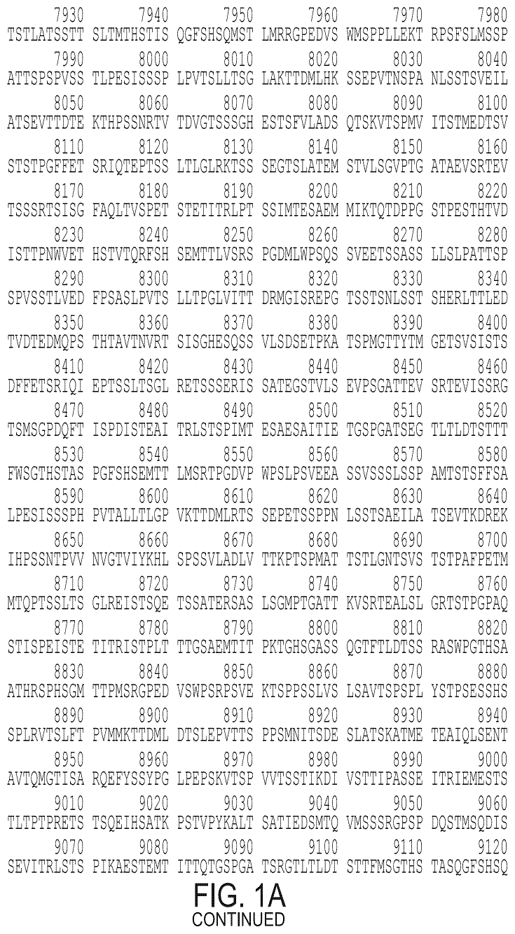

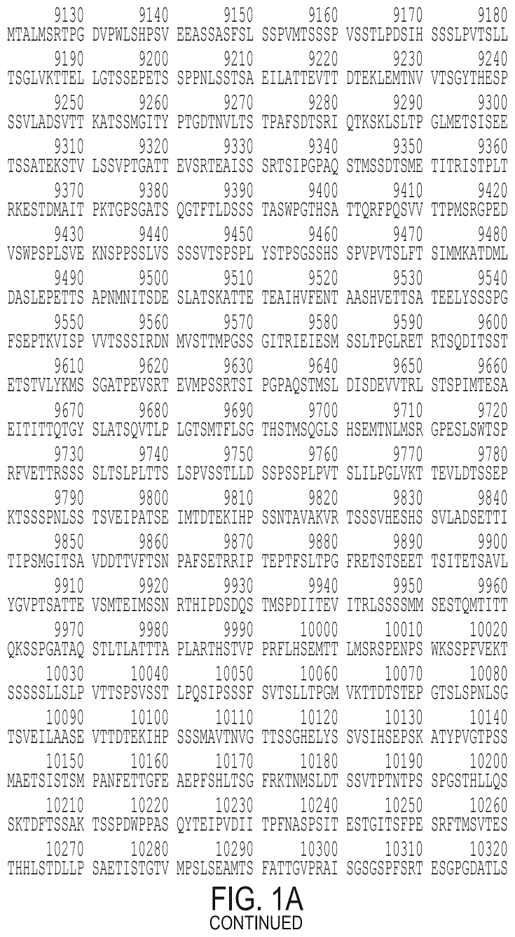

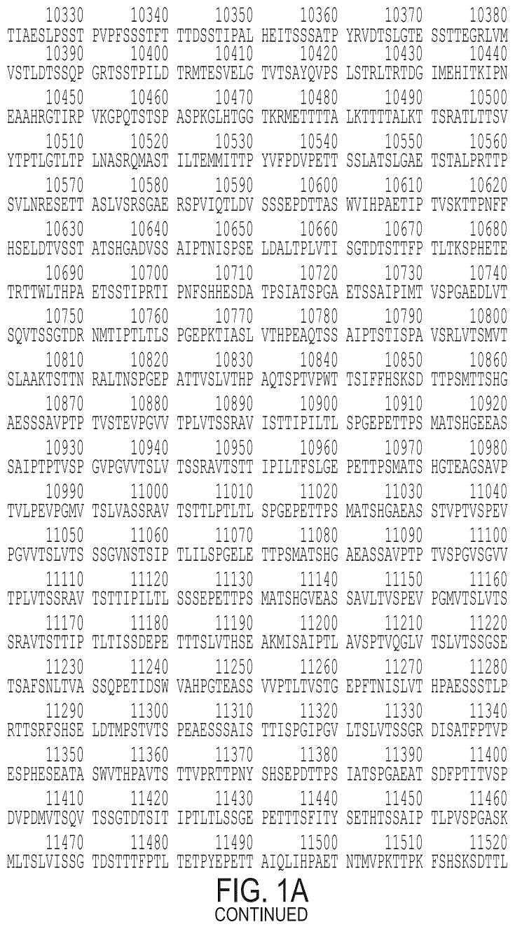

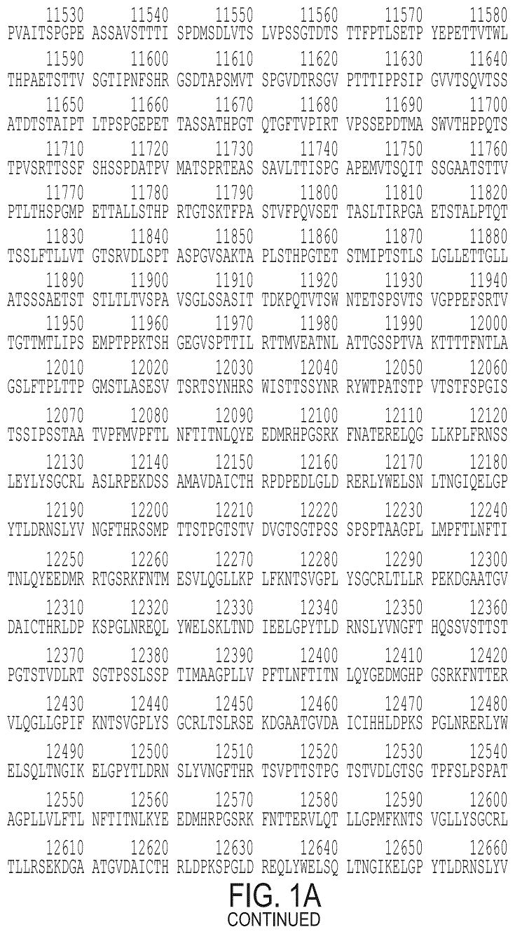

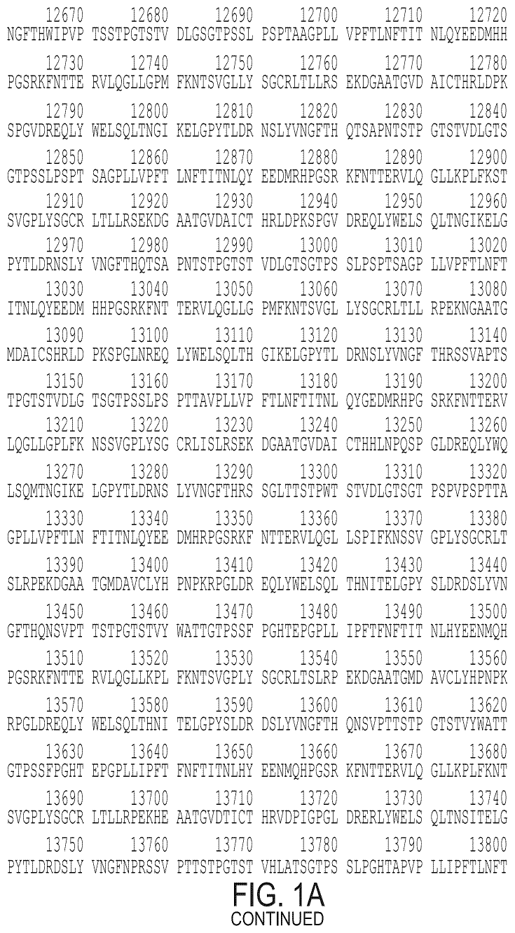

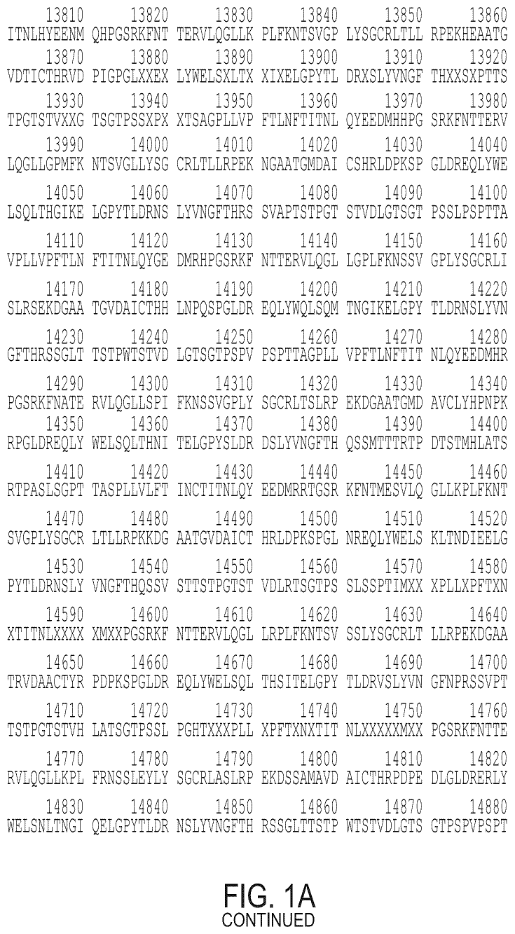

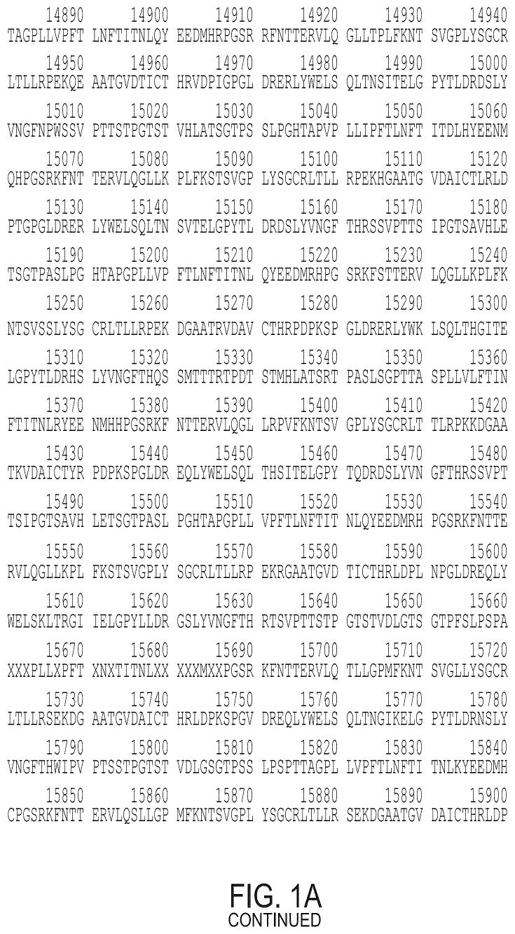

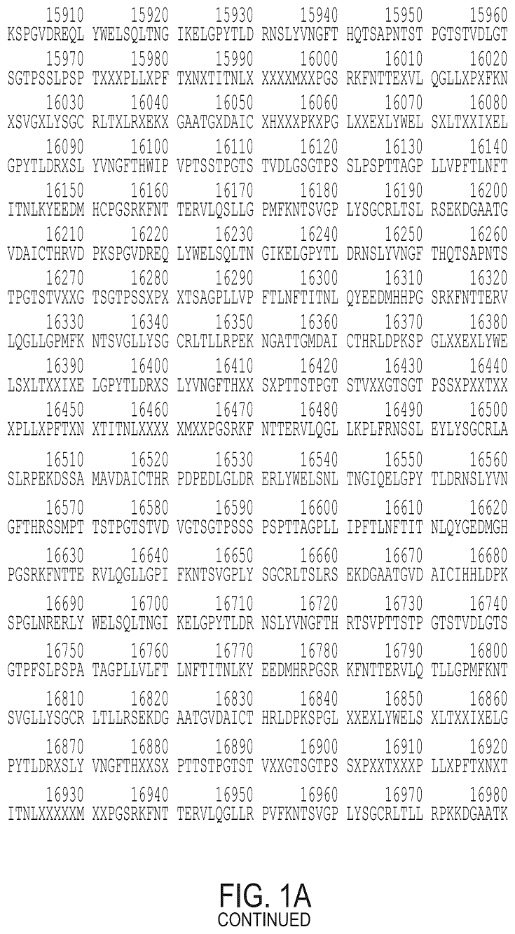

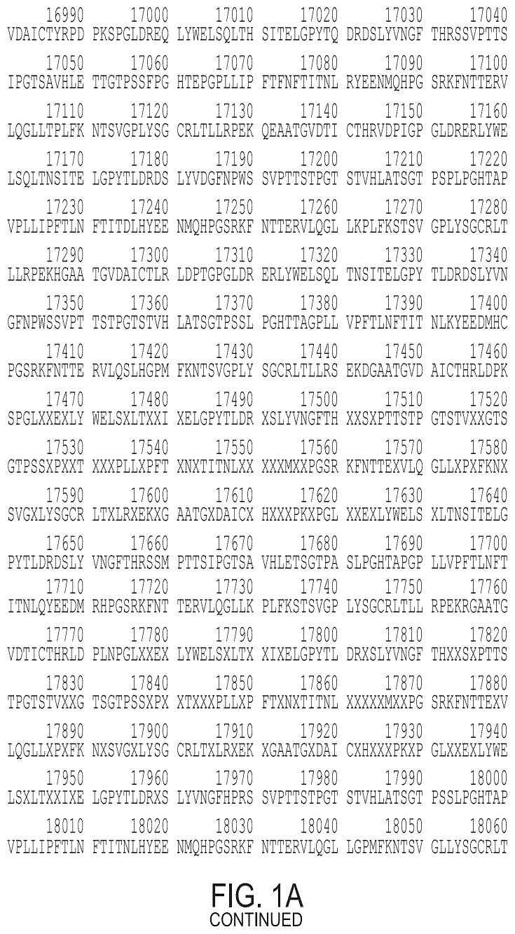

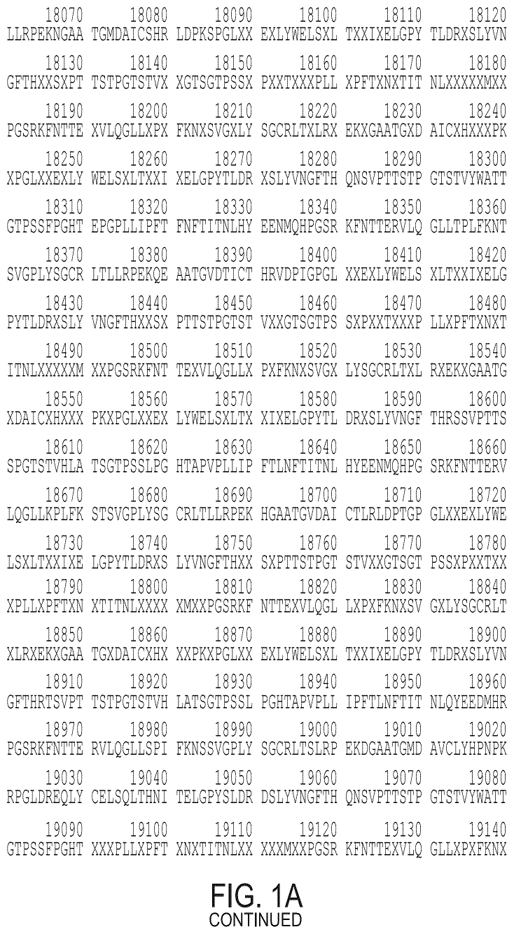

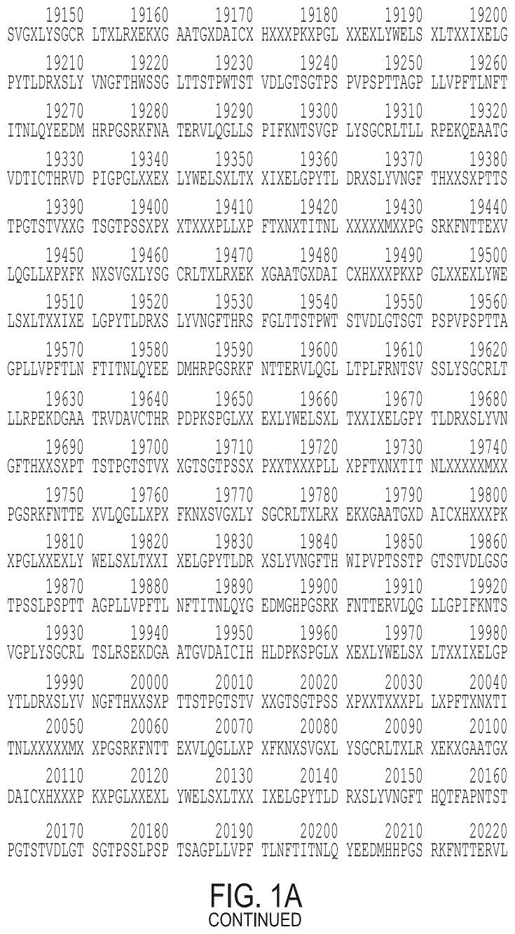

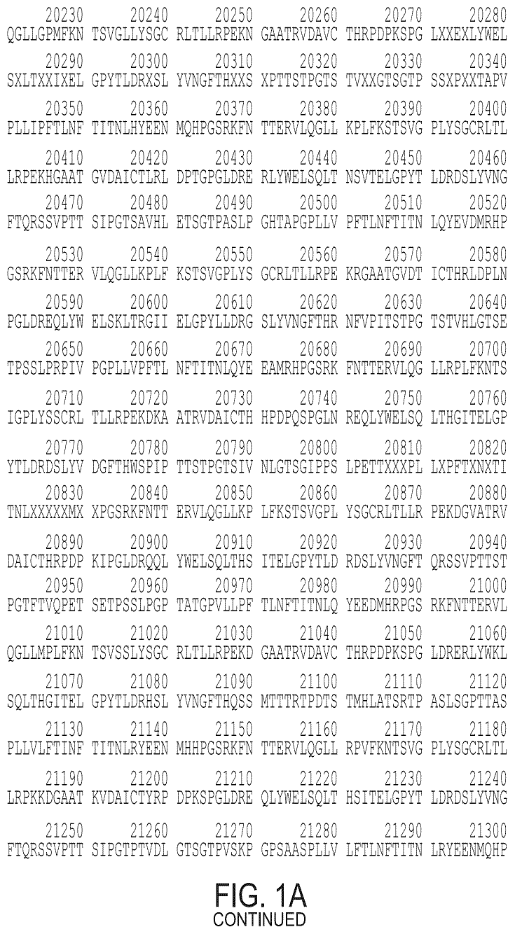

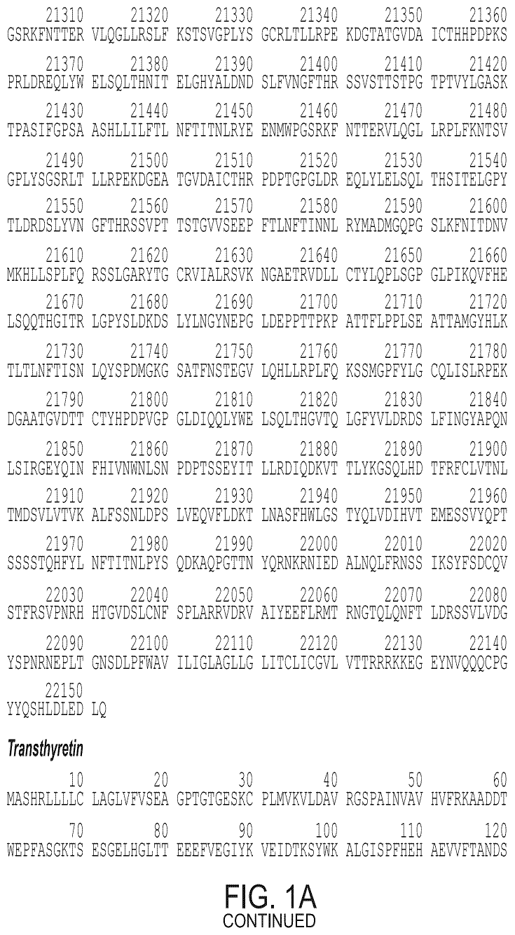

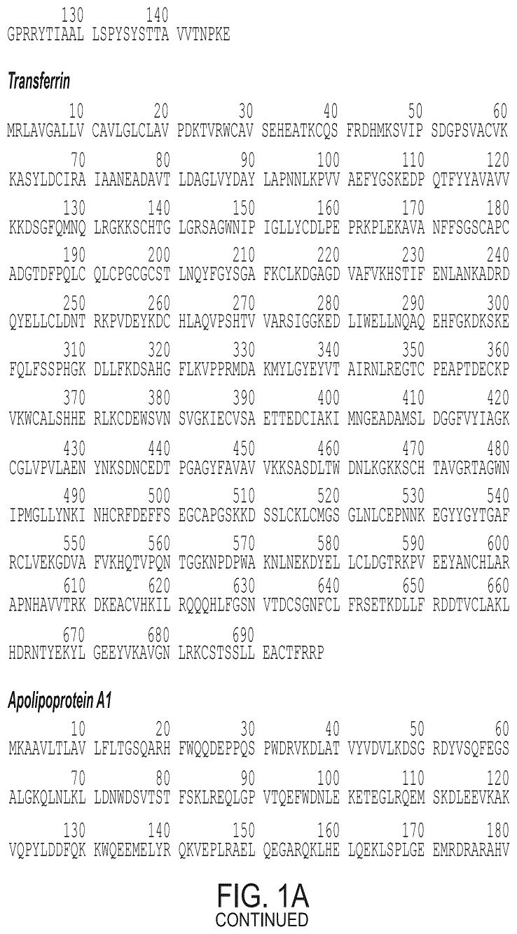

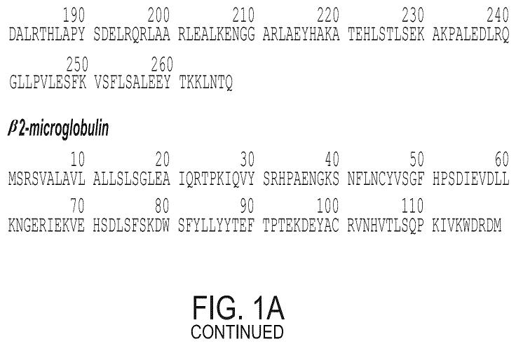

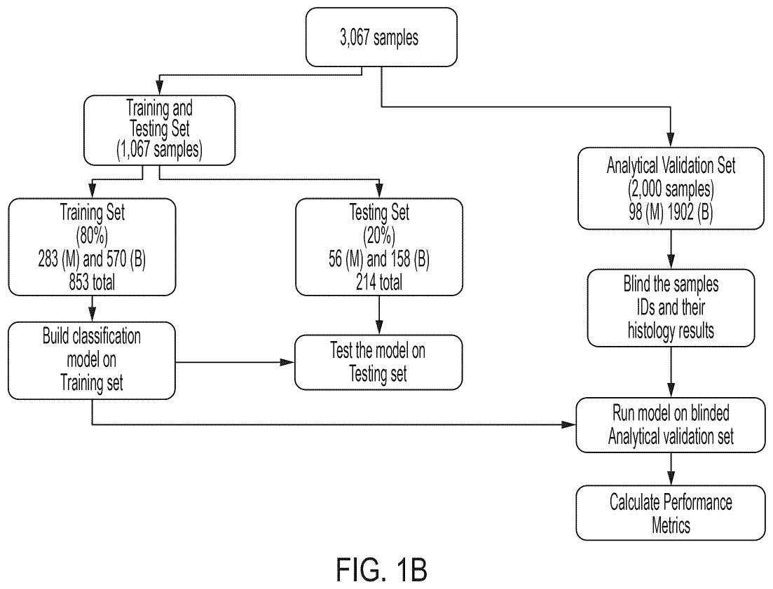

One exemplary biomarker present in the panel of the invention is FSH. FSH is a 128 amino acid protein (NCBI Accession number NP_000501). The amino acid sequence of an exemplary FSH polypeptide is set forth in FIG. 1. Antibodies to FSH can be made using any method well known in the art, or can be purchased from, for example, Santa Cruz Biotechnology, Inc. (e.g., Catalog Number sc-57149) (www.scbt.com, Santa Cruz, CA). In aspects of the invention, FSH is upregulated in subjects with ovarian cancer as compared to subjects that do not have ovarian cancer.

### Human Epididymis Protein 4 (HE4)

One exemplary biomarker present in the panel of the invention is HE4. HE4 is a 124 amino acid protein (NCBI Accession number NP_006094). The amino acid sequence of an exemplary HE4 polypeptide is set forth in FIG. 1. Antibodies to HE4 can be made using any method well known in the art, or can be purchased from, for example, Santa Cruz Biotechnology, Inc. (Catalog Number sc-27570) (www.scbt.com, Santa Cruz, CA). In aspects of the invention, HE4 is upregulated in subjects with ovarian cancer as compared to subjects that do not have ovarian cancer.

### Cancer Antigen 125 (CA125)

One exemplary biomarker present in the panel of the invention is CA125. CA125 is a 22152 amino acid protein (Swiss-Prot Accession number Q8WXI7). The amino acid sequence of an exemplary CA125 polypeptide is set forth in FIG. 1. Antibodies to CA125 can be made using any method well known in the art, or can be purchased from, for example, Santa Cruz Biotechnology, Inc. (Catalog Number sc-52095) (www.scbt.com, Santa Cruz, CA). In aspects of the invention, CA125 is upregulated in subjects with ovarian cancer as compared to subjects that do not have ovarian cancer.

### Transthyretin (Prealbumin)

Another exemplary biomarker present in the panel of the invention is a form of pre-albumin, also referred to herein as transthyretin. Transthyretin is a 147 amino acid protein (Swiss Prot Accession number P02766). The amino acid sequence of an exemplary transthyretin polypeptide is set forth in FIG. 1. Antibodies to transthyretin can be made using any method well known in the art, or can be purchased from, for example, Santa Cruz Biotechnology, Inc. (Catalog Number sc-13098) (www.scbt.com, Santa Cruz, CA). In aspects of the invention, transthyretin is downregulated in subjects with ovarian cancer as compared to subjects that do not have ovarian cancer.

### Transferrin

Transferrin is another exemplary biomarker of the panel of biomarkers of the invention. Transferrin is a 698 amino acid protein (UniProtKB/TrEMBL Accession number Q06AH7). The amino acid sequence of an exemplary transferring polypeptide is set forth in FIG. 1. Antibodies to transferrin can be made using any method well known in the art, or can be purchased from, for example, Santa Cruz Biotechnology, Inc. (Catalog Number sc-52256) (www.scbt.com, Santa Cruz, CA). In aspects of the invention, transferrin is downregulated in subjects with ovarian cancer as compared to subjects that do not have ovarian cancer.

### Apolipoprotein A1

Apolipoprotein A1, also referred to herein as “ApoA1,” is another exemplary biomarker in the panel of biomarkers of the invention. ApoA1 is a 267 amino acid protein (Swiss Prot Accession number P02647). The amino acid sequence of an exemplary ApoA1 is set forth in FIG. 1. Antibodies to Apolipoprotein A1 can be made using any method well known in the art, or can be purchased from, for example, Santa Cruz Biotechnology, Inc. (Catalog Number sc-130503) (www.scbt.com, Santa Cruz, CA). In aspects of the invention, ApoA1 is downregulated in subjects with ovarian cancer as compared to subjects that do not have ovarian cancer.

### β2 Microglobulin

One exemplary biomarker that is useful in the methods of the present invention is β2-microglobulin. β-microglobulin is described as a biomarker for ovarian cancer in US provisional patent publication 60/693,679, filed Jun. 24, 2005 (Fung et al.). The mature form of β2-microglobulin is a 99 amino acid protein derived from an 119 amino acid precursor (GI:179318; SwissProt Accession No. P61769). The amino acid sequence of an exemplary β-2-microglobulin polypeptide is set forth in FIG. 1. The mature form of β-2-microglobulin consist of residues 21-119 of the β-2-microglobulin set forth in FIG. 1. β2-microglobulin is recognized by antibodies. Such antibodies can be made using any method well known in the art, and can also be commercially purchased from, e.g., Abcam (catalog AB759) (www.abcam.com, Cambridge, MA). In aspects of the invention, β2-microglobulin is upregulated in subjects with ovarian cancer as compared to subjects that do not have ovarian cancer.

### Biomarkers and Different Forms of a Protein

Proteins frequently exist in a sample in a plurality of different forms. These forms can result from pre- and/or post-translational modification. Pre-translational modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulphonation and acetylation. When detecting or measuring a protein in a sample, any or all of the forms may be measured to determine the level of biomarker or a form of interest is measured. The ability to differentiate between different forms of a protein depends upon the nature of the difference and the method used to detect or measure the protein. For example, an immunoassay using a monoclonal antibody will detect all forms of a protein containing the epitope and will not distinguish between them. However, a sandwich immunoassay that uses two antibodies directed against different epitopes on a protein will detect all forms of the protein that contain both epitopes and will not detect those forms that contain only one of the epitopes. Distinguishing different forms of an analyte or specifically detecting a particular form of an analyte is referred to as “resolving” the analyte.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology combines mass spectrometry with immunoassay. For example, a biospecific capture reagent (e.g., an antibody, aptamer, Affibody, and the like that recognizes the biomarker and other forms of it) is used to capture the biomarker of interest. In embodiments, the biospecific capture reagent is bound to a solid phase, such as a bead, a plate, a membrane or an array. After unbound materials are washed away, the captured analytes are detected and/or measured by mass spectrometry. This method will also result in the capture of protein interactors that are bound to the proteins or that are otherwise recognized by antibodies and that, themselves, can be biomarkers. Various forms of mass spectrometry are useful for detecting the protein forms, including laser desorption approaches, such as traditional MALDI or SELDI, electrospray ionization, and the like.

Thus, when reference is made herein to detecting a particular protein or to measuring the amount of a particular protein, it means detecting and measuring the protein with or without resolving various forms of protein. For example, the step of “detecting β-2 microglobulin” includes measuring β-2 microglobulin by means that do not differentiate between various forms of the protein (e.g., certain immunoassays) as well as by means that differentiate some forms from other forms or that measure a specific form of the protein.

### Detection of Biomarkers for Ovarian Cancer

The biomarkers of this invention can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the invention include, but are not limited to, optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described infra.

### Detection by Immunoassay

In particular embodiments, the biomarkers of the invention are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In aspects of the invention, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In aspects of the invention, a sample is analyzed by means of a protein biochip (also known as a protein microarray). Such biochips are useful in high-throughput low-cost screens to identify alterations in the expression or post-translation modification of a polypeptide of the invention, or a fragment thereof. In embodiments, a protein biochip of the invention binds a biomarker present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In some embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy or liquid biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, CA), Zyomyx (Hayward, CA), Packard BioScience Company (Meriden, CT), Phylos (Lexington, MA), Invitrogen (Carlsbad, CA), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy or liquid biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 4° C. in 15 mM NaCl, 1.5 mM trisodium citrate, and 0.1% SDS. In other embodiments, wash steps will occur at 6° C. in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In aspects of the invention, the biomarkers of this invention are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using a server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MS” analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quatemary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Methods of the Invention

Panels comprising biomarkers of the invention are used to characterize a pelvic mass in a subject to determine whether the subject has an adnexal mass which is benign or of indeterminate risk. In certain embodiments, panels of the invention are used to select a course of treatment for a subject (e.g., a conservative course of treatment which avoids or delays surgical intervention). The phrase “ovarian cancer status” includes any distinguishable manifestation of the disease, including non-disease. For example, ovarian cancer status includes, without limitation, the presence or absence of disease (e.g., ovarian cancer v. non-ovarian cancer), the risk of developing disease, the stage of the disease, the progression of disease (e.g., progress of disease or remission of disease over time), prognosis, the effectiveness or response to treatment of disease, and the determination of whether a pelvic mass is malignant of benign, symptomatic or asymptomatic. Based on this status, further procedures may be indicated, including additional diagnostic tests or therapeutic procedures or regimens. In aspects of the invention, the biomarkers of the invention can be used in diagnostic tests to identify early stage ovarian cancer in a subject.

In some embodiments, the panel of biomarkers include, but are not limited to, Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH).

In some embodiments, the characterization of a panel of biomarkers in a biological sample from a subject determines a score that identifies that subject as having a benign adnexal mass or having an adnexal mass having an indeterminate risk of malignancy. In some embodiments, the range of scores indicating an adnexal mass having an indeterminate risk of maliganacy is further subdivided into scores indicating an adnexal mass having an intermediate risk of malignancy and scores indicating an adnexal mass having a high risk of malignancy. In some embodiments, the range of scores may be further subdivided.

In many embodiments the score is normalized to a 10 point scale. In some embodiments, the method further includes determining, that the subject has a benign adnexal mass where the score is between 0.0 and less than 2.5; determining, that the subject has an adnexal mass with an intermediate risk of malignancy where the score is between 2.5 and less than 5.0; and determining, that the subject has an adnexal mass with a high risk of malignancy where the score is between 5.0 and 10.0.

In some embodiments, the characterization of a first panel of markers determines a first score. In some embodiments, a subject identified by the first score with an intermediate risk of developing or having ovarian cancer is selected for further characterization with one or more panels of biomarkers. In some embodiments, the characterization of a second panel of markers determines a second score. In some embodiments, the characterization of a third panel of markers determines a third score. In many embodiments, each of the first, second, or third score may indicate a benign adnexal mass, or an adnexal mass having a indeterminate risk of malignancy. In some embodiments, the range of scores for each of the first, second, or third score, indicating an adnexal mass having an indeterminate risk of maliganacy is further subdivided into scores indicating an adnexal mass having an intermediate risk of malignancy and scores indicating an adnexal mass having a high risk of malignancy. In some embodiments, the range of scores may be further subdivided.

In some embodiments, a biological sample from a subject is further characterized by detecting whether the subject has one or more mutations in one or more germline and/or somatic markers. In some embodiments, the germline and/or somatic markers are associated with breast and/or ovarian cancer. In some embodiments, the presence of one or more mutations in one or more breast and/or ovarian cancer markers identifies a subject as in need of therapeutic intervention having a higher [increased] cancer risk relative to a subject that does not have one of these markers. In some embodiments, aberrant methylation of one or more breast and/or ovarian cancer markers identifies a subject as in need of therapeutic intervention having a higher [increased] cancer risk relative to a subject that does not have aberrant methylation of one of these markers. In some embodiments, the aberrant methylation of one or more breast and/or ovarian cancer markers is hypermethylation. In some embodiments, the aberrant methylation of one or more of the above breast and/or ovarian cancer markers is hypomethylation.

In some embodiments, the methods disclosed herein further include characterizing one or more clinical markers of ovarian cancer risk in the subject, wherein the one or more clinical biomarkers are selected from group consisting of age, pre-menopausal status, post-menopausal status, ethnicity, pathology, adnexal mass diagnosis, family history, physical examination, imaging results, and/or history of smoking, wherein the one or more clinical biomarkers further identifies the subject as having a low or high cancer risk. In some embodiments, the subject is diagnosed with an adnexal mass. In some embodiments, the subject is diagnosed with an asymptomatic adnexal mass. In some embodiments, the subject is diagnosed with a symptomatic adnexal mass. In some embodiments, the subject is pre-menopausal. In some embodiments, the subject is post-menopausal.

The method includes a diagnostic measurement (e.g., screening assay or detection assay) in a biological sample obtained from the subject suffering from or susceptible to ovarian cancer. In some embodiments, the diagnostic measurement characterizes markers in a biological sample. In some embodiments, the biological sample is serum. In some embodiments, one or more markers are characterized by detecting cell-free tumor DNA (cftDNA). In some embodiments, a panel of markers are bound to a separate capture reagent. In some embodiments, the capture reagents are attached to a solid support. In some embodiments, the solid support is a plate, chip, beads, microfluidic platform, membrane, planar microarray, or suspension array. In some embodiments, the capture reagent is an antibody, aptamer, Affibody, hybridization probe and/or fragments thereof each capture reagent specifically binds to one of the markers. In some embodiments, the markers are characterized by immunoassay, sequencing and/or nucleic acid microarray. In some embodiments, the sequencing is next-generation sequencing (NGS) or Sanger sequencing. In some embodiments, the immunoassay comprises affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay.

The correlation of test results with ovarian cancer involves applying a classification algorithm of some kind to the results to generate the status. The classification algorithm may be as simple as determining whether or not the amounts of the markers or a combination of the markers listed in Table 1 are above or below a particular cut-off number. When multiple biomarkers are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

### Biomarkers of the Invention

Individual biomarkers are useful diagnostic biomarkers. In addition, as described in the examples, it has been found that a specific combination of biomarkers provides greater predictive value of a particular status than any single biomarker alone, or any other combination of previously identified biomarkers. Specifically, the detection of a plurality of biomarkers in a sample can increase the sensitivity, accuracy and specificity of the test.

Each biomarkers described herein can be differentially present in ovarian cancer, and, therefore, each is individually useful in aiding in the determination of ovarian cancer status. The method involves, first, measuring the selected biomarker in a subject, sample using any method well known in the art, including but not limited to the methods described herein, e.g. capture on a SELDI biochip followed by detection by mass spectrometry and, second, comparing the measurement with a diagnostic amount or cut-off that distinguishes a positive ovarian cancer status from a negative ovarian cancer status. The diagnostic amount represents a measured amount of a biomarker above which or below which a subject is classified as having a particular ovarian cancer status. For example, if the biomarker is up-regulated compared to normal during ovarian cancer, then a measured amount above the diagnostic cutoff provides a diagnosis of ovarian cancer. Alternatively, if the biomarker is down-regulated during ovarian cancer, then a measured amount below the diagnostic cutoff provides a diagnosis of ovarian cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different ovarian cancer statuses, as was done here, and drawing the cut-off to suit the diagnostician's desired levels of specificity and sensitivity.

The biomarkers of this invention (used alone or in combination) show a statistical difference in different ovarian cancer statuses of at least p≤0.05, p≤10⁻², p≤10⁻³, p≤10⁻⁴, or p≤10⁻⁵. Diagnostic tests that use these biomarkers alone or in combination show a sensitivity and specificity of at least 75%, at least 80%, at least 85%, at least 90%, at least 95%, at least 98%, or about 100%.

### Determining Course (Progression/Remission) of Disease

In one embodiment, this invention provides methods for monitoring or determining the course of disease in a subject. Disease course refers to changes in disease status over time, including disease progression (worsening) and disease regression (improvement). Over time, the amounts or relative amounts (e.g., the pattern) of the biomarkers change. Accordingly, this method involves measuring or characterizing a panel of biomarkers in a biological sample from a subject during at least two different time points, e.g., a first time and a second time, and comparing the change in amounts, if any. The course of disease (e.g., during treatment) is determined based on these comparisons.

In some embodiments, the panel of biomarkers include, but are not limited to, Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH).

In some embodiments, methods for monitoring or determining the course of disease in a subject further characterizes one or more clinical biomarkers of ovarian cancer risk in the subject, wherein the one or more clinical biomarkers are selected from group consisting of age, pre-menopausal status, post-menopausal status, ethnicity, pathology, adnexal mass diagnosis, family history, physical examination, imaging results, and/or history of smoking, wherein the one or more clinical biomarkers further identifies the subject as having a low or high cancer risk. In some embodiments, a subject diagnosed with an adnexal mass having a low or intermediate risk of developing ovarian cancer is monitored for disease progression (i.e., high risk status). In some embodiments, the subject is diagnosed with an asymptomatic adnexal mass. In some embodiments, the subject is diagnosed with a symptomatic adnexal mass. In some embodiments, the subject is pre-menopausal. In some embodiments, the subject is post-menopausal.

The method includes a diagnostic measurement (e.g., screening assay or detection assay) in a biological sample obtained from the subject suffering from or susceptible to ovarian cancer. In some embodiments, the diagnostic measurement characterizes markers in a biological sample. In some embodiments, the biological sample is serum. In some embodiments, one or more markers are characterized by detecting cell-free tumor DNA (cftDNA). In some embodiments, a panel of markers are bound to a separate capture reagent. In some embodiments, the capture reagents are attached to a solid support. In some embodiments, the solid support is a plate, chip, beads, microfluidic platform, membrane, planar microarray, or suspension array. In some embodiments, the capture reagent is an antibody, aptamer, Affibody, hybridization probe and/or fragments thereof each capture reagent specifically binds to one of the markers. In some embodiments, the markers are characterized by immunoassay, sequencing and/or nucleic acid microarray. In some embodiments, the sequencing is next-generation sequencing (NGS) or Sanger sequencing. In some embodiments, the immunoassay comprises affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay.

The diagnostic measurement in the method can be compared to samples from healthy, normal controls; in a pre-disease sample of the subject; or in other afflicted/diseased patients to establish the treated subject's disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject or CT scan) is determined prior to beginning treatment as described; this measurement can then be compared to a measurement in the subject after the treatment commences and/or during the course of treatment to determine the efficacy of (monitor the efficacy of) the disease treatment. In some embodiments, efficacy of the disease treatment can be performed with antibody marker analysis and/or interferon-gamma (IFN-γ) ELISPOT assays.

### Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the differential presence or absence in a test subject of the biomarkers or a combination of the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Subject Management

In certain embodiments, the methods of the invention involve managing subject treatment based on the status. For patient's whose condition is determined to be low risk such management includes, for example, watchful waiting, which may involve periodically retesting the patient to determine whether levels of biomarkers have changed, and whether such change is indicative of an increased risk of ovarian cancer. In some embodiments, such testing may indicate that the subject should be referred, for example, to a gynecologic oncologist. For example, if a physician makes a diagnosis of ovarian cancer, then a certain regime of treatment, such as prescription or administration of therapeutic agent might follow. Alternatively, a diagnosis of non-ovarian cancer or non-ovarian cancer might be followed with further testing to determine a specific disease that might the patient might be suffering from. Also, if the diagnostic test gives an inconclusive result on ovarian cancer status, repeated biomarker testing may be called for.

In one embodiment, the diagnosis may be determining if a pelvic mass is benign or malignant. If the diagnosis is malignant, a gynecologic oncologist may be chosen to perform the surgery. In contrast, if the diagnosis is benign, watchful waiting and periodic re-testing of the subject may be appropriate.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

### Hardware and Software

The any of the methods described herein, the step of correlating the measurement of the biomarker(s) with ovarian cancer can be performed on general-purpose or specially-programmed hardware or software (e.g., through a computer-implemented method).

In aspects, the analysis is performed by a software classification algorithm (e.g., an artificial neural network). The analysis of analytes by any detection method well known in the art, including, but not limited to the methods described herein, will generate results that are subject to data processing. Data processing can be performed by the software classification algorithm. Such software classification algorithms are well known in the art and one of ordinary skill can readily select and use the appropriate software to analyze the results obtained from a specific detection method.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

For example, analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. Exemplary software includes, but is not limited to, Ciphergen's ProteinChip® software, in which data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, for example, as part of Ciphergen's ProteinChip® software package, that can automate the detection of peaks. This software functions by identifying signals having a signal-to-noise ratio above a selected threshold and labeling the mass of the peak at the centroid of the peak signal. In embodiments, many spectra are compared to identify identical peaks present in some selected percentage of the mass spectra. One version of this software clusters all peaks appearing in the various spectra within a defined mass range, and assigns a mass (N/Z) to all the peaks that are near the mid-point of the mass (M/Z) cluster.

In aspects, software used to analyze the data can include code that applies an algorithm to the analysis of the results (e.g., signal to determine whether the signal represents a peak in a signal that corresponds to a biomarker according to the present invention). The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

### Classification Algorithms for Qualifying Adnexal Mass Status

In some embodiments, data derived from the assays (e.g., ELISA assays) that are generated using samples such as “known samples” can then be used to “train” a classification model. A “known sample” is a sample that has been pre-classified. The data that are derived from the spectra and are used to form the classification model can be referred to as a “training data set” or “training set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased). In some embodiments, the training data set may be segregated into one or more subsets, preferably corresponding to classes (e.g., a data set corresponding to benign ovarian tumors and/or adnexal masses, and a data set corresponding to malignant ovarian tumors and/or adnexal masses).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories (e.g., benign ovarian tumors and/or adnexal masses or malignant ovarian tumors and/or adnexal masses) are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART-classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer, on any suitable computing device, or on one or more suitable computing devices, such as, for example, a plurality of suitable computing devices or cloud computing devices. Suitable digital computers, or computing devices, include, but are not limited to, micro, mini, or large computers using any standard or specialized operating system, such as a Unix, Windows™ or Linux™ based operating system. The digital computer or computing device that is used may be physically separate from the analysis device, such as a mass spectrometer, that is used to create the spectra of interest, or it may be coupled to the analysis device or mass spectrometer.

The training data set and the classification models according to embodiments of the invention can be embodied by computer code that is executed or used by a digital computer and/or computing device. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for ovarian cancer. The classification algorithms, in turn, form the base for diagnostic tests by providing diagnostic values (e.g., cut-off points) for biomarkers used singly or in combination.

In some embodiments, the methods of the invention include classifying a subject's risk of having ovarian cancer. In some embodiments, the method includes receiving, by at least one processor, a signal representing a marker spectrum peak detected for each marker of a panel. In some embodiments, one or more panels are used. In some embodiments, the panel includes, but is not limited to, markers Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH).

In some embodiments, the method includes receiving, by at least one processor, a panel signal representing a marker spectrum peak detected for each marker of a panel comprising markers Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH).

In some embodiments, the method utilizes, by the at least one processor, a first stage cancer risk classifier to predict a cancer risk classification score representative of a predicted risk of developing ovarian cancer, the cancer risk classification score being based on learned risk classification parameters and the first panel signal. In some embodiments, the method determines, by the at least one processor, a cancer risk level associated with the cancer risk classification score, the cancer risk level selected from one of at least the selection comprising low risk, intermediate risk and high risk. In some embodiments, the method generates, by the at least one processor, a cancer risk level prediction at a computing device associated with a care provider indicative of the cancer risk level of the subject.

In many embodiments the cancer risk score or cancer risk classification score is normalized to a 10 point scale. In some embodiments, the method further includes determining, by the at least one processor and/or computing device, that the subject has a benign adnexal mass where the cancer risk classification score is between 0.0 and less than 2.5; determining, by the at least one processor and/or computing device, that the subject has an adnexal mass with an intermediate risk of malignancy where the cancer risk classification score is between 2.5 and less than 5.0; and determining, by the at least one processor, that the subject has an adnexal mass with a high risk of malignancy where the cancer risk classification score is between 5.0 and 10.0.

### Characterization of Non-Malignant or Asymptomatic Ovarian Tumor or Adnexal Mass

In many embodiments, the methods of the present disclosure include selecting a subject, or providing a selected subject, where the selected subject is selected by pre-characterization, or characterizing beforehand that the subject has a non-malignant, or asymptomatic ovarian tumor or adnexal mass. Preferably, such pre-characterization or characterzation is performed by a medical professional, in a clinical or other professional setting. Such pre-characterization or characterization may be conducted through the use of any appropriate assay or screen, such as, for example, imaging or biomarker screening. In an embodiment, the imaging is transvaginal ultrasonography (TVUS). In an exemplary embodiment, a medical professional performs or provides the pre-characterization or characterization that a given ovarian tumor or adnexal mass is asymptomatic or non-malignant through TVUS imaging and/or monitoring over the course of 6 months, without an increase in the size of the tumor or mass. In an embodiment, the biomarker screening is CA125 screening, or HE4 screening.

### Conservative Management of Adnexal Masses

In another aspect, the invention provides a method of conservative management of an adnexal mass in a selected subject. Ovarian malignancy is rare, even amongst women with an adnexal mass. A substantial portion of such masses resolve on subsequent imaging. Therefore, a method of conservative management of adnexal masses is needed. In some embodiments, the conservative management includes the measurement or characterization of a panel of biomarkers, such as those listed in Table 1. This method may include the selection of a particular subject population, but in other cases, may be used in any subject having an adnexal mass. In some embodiments, the subject is selected on the basis of having at least on contraindication to surgical intervention, preferably where such contraindications include, but are not limited to, a comorbidity which precludes surgical intervention, the desire to maintain fertility in the subject, or a risk or significant risk of harming fertility in the subject, or characteristics of the adnexal mass, such as size or visibility of the adnexal mass, or pain or discomfort or lack thereof in the adnexal mass. In some embodiments, the panel of biomarkers is used to determine a score, preferably where the score identifies the subject as having a benign adnexal mass, or having an adnexal mass having an indeterminate risk of malignancy. In some embodiments, any method disclosed herein may be used to determine this score, including any computer-implemented method using any classification engine, machine learning engine, or artificial neural network disclosed herein. In some embodiments, when the score identifies the subject as having a benign adnexal mass, the subject may be directed to not seek surgical intervention. In some embodiments, when the score identifies the subject as having a benign adnexal mass, the adnexal mass may be subject to further conservative treatment or management, preferably where such conservative treatment or management includes the avoidance or delay of surgical intervention.

### Artificial Neural Networks

In another aspect, the invention provides a computer implemented method of assessing or diagnosing ovarian cancer or the risk of ovarian in a selected subject (e.g., a subject having an adnexal mass previously determined to be non-malignant or asymptomatic), preferably utilizing a classification model or system, more preferably where the classification model or system is an artificial neural network. In some embodiments, the computer implemented method includes the use of one or more computing devices. In some embodiments, the computer implemented method involves the measurement or characterization of panels of biomarkers of the present disclosure, for example, such as those biomarkers identified in Table 1. In some embodiments, the one or more computing devices receive a plurality of signals, each signal representing a value of a biomarker from a panel of biomarkers, such as those biomarkers identified in Table 1. In such embodiments, the signal may be derived through any analysis method of the present disclosure, such as, but not limited to, a photometric assay and/or mass spectrometry. Additional data may be inputted into the one or more computing devices, such as age of the selected subject, or menopausal status of the selected subject. In some embodiments, the plurality of signals, the age of the selected subject, and/or the menopausal status of the selected subject may be provided as input to the artificial neural network.

Artificial neural networks may be structured to include a plurality of nodes, preferably ordered into a plurality of layers. Each node of the artificial neural network connects to one or more other nodes of the artificial neural network, and each such connection preferably includes one or more of a weight, a bias, and/or an activation function, each of which may be a factor in determining the output from a given node to a connected node. Weights may indicate the importance of specific nodes or connections between nodes, and in an exemplary embodiment, a higher weight may be assigned to detection of malignant samples of ovarian tumors or adnexal masses. Such layers may include an input layer, which includes one or more input nodes, one or more hidden layers, each of which includes one or more hidden nodes, and an output layer, which includes one or more output nodes. The input nodes may be configured to accept input (e.g., a plurality of signals representing a biomarker panel, an age of a selected subject, and/or a menopausal status of a selected subject. The input nodes may then connect to other nodes of the artificial neural network, such as the hidden nodes, which may then in turn connect to the output nodes. In an exemplary embodiment, the output nodes and/or output layer, provide output representing the probability of a malignant ovarian tumor or adnexal mass, preferably through a cancer risk classification score. In an exemplary embodiment, the artificial neural network is a feed-forward neural network, or a deep feed-forward neural network, in which the connections between nodes do not form loops or cycles.

In some embodiments, the number of input nodes equals the number of types of input data. For example, where the input data includes a panel of seven biomarkers, such as biomarkers selected from Table 1, an age value of the selected subject, and a menopausal status of the selected subject, the artificial neural network includes nine input nodes, each corresponding to one of the previous types of input data. In an exemplary embodiment, the data from the input nodes is fed to one or more hidden layers or hidden nodes, preferably where each hidden layer or hidden node has a different weight, bias, and/or activation function. In an embodiment, the output from the hidden layers is then inputted into one or more output nodes, preferably where each output node represents a different class, or the probability of a different class. For example, an artificial neural network of the present disclosure may include two output nodes, one output node corresponding to classification of a benign ovarian tumor or adnexal mass, and one output node corresponding to an indeterminate risk of malignancy of an ovarian tumor or adnexal mass. In an exemplary embodiment, the artificial neural network may use the softmax function to assign one or more of the output node values. In an embodiment, the value of the output nodes may be combined into a cancer risk classification score.

In many embodiments, the artificial neural network is trained through the use of one or more training data sets. Preferably such training improves the output of the artificial neural network over time, ideally without overfitting or underfitting. Improvements in the output of the artificial neural network include, for example, increases in sensitivity, specificity, negative predictive value, and/or positive predictive value. In an exemplary embodiment, the training data set includes at least a data set corresponding to a class representing benign ovarian tumors or adnexal masses, and a data set corresponding to a class representing malignant ovarian tumors or adnexal masses. In such embodiments, the training data sets may be derived through historical data from ovarian tumors or adnexal masses in which the status of the ovarian tumor or adnexal mass was verified through surgical histology or another clinical assay. In some embodiments, training data sets may be balanced or unbalanced. In a balanced training set, the samples in the training data are roughly evenly distributed amongst the classes, or the number of samples in each classification are approximately the same. Conversely, in an unbalanced training set, the samples in the training set are not balanced. Training sets may be naturally balanced, or balanced through the creation of artificial or synthetic samples, preferably where such artificial or synthetic samples are created near the decision boundary. Training of the artificial neural networks of the present disclosure may include any technique known in the art, including those for reducing overfitting and underfitting. Such techniques may include, for example, the synthetic minority oversampling technique (SMOTE), or variants thereof, or the node dropout technique.

Since a training set with a low number of positives (e.g., a low number of malignant samples) may result in an artificial neural network with high specificity, but low sensitivity, a training set including at least a set number of positive samples (e.g., malignant samples) may be used. In some embodiments, the training set may include at least 100 positive samples. In some embodiments, the training set may include 100-500 positive samples. In some embodiments, the training set may include more than 500 positive samples.

### Serial Monitoring

In an aspect, the methods of the present disclosure may be performed over at least two or more time points, in order to monitor a subject's risk of having ovarian cancer. In some embodiments, the methods of the present disclosure are repeated once every 1, 2, 3, 4, 5, 6, 7, 8, 9, 10, 11, or 12 months, or greater than 12 months.

Significant increases in the cancer risk classification score, risk probability, or other ovarian risk indicator provided by the methods of the present disclosure between two or more consecutive performances of the method of the present disclosure may require intervention by a medical professional. In some embodiments, the subject is recommended for clinical follow-up when a score change of greater than 2.25 between two successive time points in the plurality of time points is detected.

### Kits for Detection of Biomarkers for Ovarian Cancer

In another aspect, the invention provides kits for aiding in the diagnosis of ovarian cancer (e.g., identifying ovarian cancer status, detecting ovarian cancer, identifying early stage ovarian cancer, selecting a treatment method for a subject at risk of having ovarian cancer, and the like), which kits are used to detect biomarkers according to the invention. In one embodiment, the kit comprises agents that specifically recognize the biomarkers or combinations of the biomarkers identified in Table 1. The kit may contain 1, 2, 3, 4, 5, or more different agents that each specifically recognize one of the biomarkers. In related embodiments, the agents are antibodies, aptamers, Affibodies, hybridization probes and/or fragments thereof.

In another embodiment, the kit comprises a solid support, such as a chip, a microtiter plate or a bead or resin having capture reagents attached thereon, wherein the capture reagents bind the biomarkers of the invention. Thus, for example, the kits of the present invention can comprise mass spectrometry probes for SELDI, such as ProteinChip® arrays. In the case of biospecific capture reagents, the kit can comprise a solid support with a reactive surface, and a container comprising the biospecific capture reagents.

The kit can also comprise a washing solution or instructions for making a washing solution, in which the combination of the capture reagent and the washing solution allows capture of the biomarker or biomarkers on the solid support for subsequent detection by, e.g. mass spectrometry. The kit may include more than type of adsorbent, each present on a different solid support.

In a further embodiment, such a kit can comprise instructions for use in any of the methods described herein, preferably with instructions for use in a selected subject (e.g., a subject having an adnexal mass previously determined to be non-malignant or asymptomatic). In embodiments, the instructions provide suitable operational parameters in the form of a label or separate insert. For example, the instructions may inform a consumer about how to collect the sample, how to wash the probe or the particular biomarkers to be detected.

In yet another embodiment, the kit can comprise one or more containers with controls (e.g., biomarker samples) to be used as standard(s) for calibration.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Analytical Validation of a Deep Neural Network Algorithm for the Detection of Ovarian Cancer

Adnexal masses are a common gynecological condition. With approximately 10% of women undergoing surgery for an adnexal mass during their lifetime, the research efforts to date have focused on tools designed to identify which of these masses are cancerous [1-2]. Ovarian cancer is the deadliest gynecological cancer, therefore prompt and correct identification of malignancies is crucial. However, the incidence of ovarian cancer is still relatively low [3]. Approximately 85% of masses in premenopausal women will be benign, so testing that can accurately differentiate malignant masses from those that require less extensive intervention and treatment is of clinical value [1].

Identification of a pelvic mass may occur during physical examination but more likely via imaging, typically with transvaginal ultrasonography (TVUS). Biopsy is usually avoided to reduce the risk of disrupting the cyst wall and allowing any potential malignant cells to disseminate [4]. When a mass shows clear indications of malignancy, the patient benefits from appropriate referral to a gynecologic oncologist for surgery, staging, and any further treatment [5].

Beyond imaging, additional methods of assessing adnexal masses include the use of biomarker-based blood tests, such as CA125 and HE4. Relying on these traditional methods to stratify the oncological risk of adnexal masses has several challenges. First, a small set of biomarkers may not be able to ascertain the physiology of certain ovarian cancers because different histologic subtypes are known to present with different biomarker patterns [6-8]. Second, the process of using a set threshold for each biomarker can become cumbersome when multiple markers are added to the analysis. Third, this process may be further complicated by the age and menopausal status of the patient which can impact the baseline, or so called ‘normal’ level of these proteins.

Machine learning-based classification models can address these limitations which is why their use in early cancer detection and risk stratification is increasing [9]. These models are capable of incorporating a long list of protein biomarkers along with clinical/health features as inputs to generate a unified score for risk assessment. However, building these models can be challenging due to the low incidence of ovarian cancer. Having a small set of positive samples for training can result in a skewed model with a high specificity but a low sensitivity. Developing a balanced classification model with high sensitivity and specificity is crucial, especially given the mortality implications of false negatives and the burden on the healthcare system and the patient of false positives.

This study describes the development and validation process used to establish test performance metrics for MIA3G, a new machine learning algorithm to assess ovarian cancer risk in patients with an adnexal mass. Powered by a robust data set inclusive of a large number of malignancies for training and testing, this algorithm has demonstrated balanced performance in a large analytical validation set.

### Example 2: Algorithm Description

The MIA3G assay is an algorithm developed with a proprietary application of machine learning methods whose purpose is to stratify women with an ovarian mass in to two categories-low or elevated risk of malignancy. The algorithm uses supervised learning with known histopathology diagnoses (malignant and non-malignant) as the labels for algorithm training. MIA3G is a classification deep feedforward neural network which utilizes the following features as inputs: age, menopausal status, and seven protein biomarker values for each patient. The neural network has multiple hidden layers each with their own weighted nodes and activation functions. The neural network is regularized using node dropout to reduce overfitting where a percentage of the nodes are randomly omitted from each hidden layer during training [10]. The final layer of the neural network has two nodes and uses the softmax function to assign a binary classification: low or elevated risk of malignancy.

### Example 3: Protein Biomarkers and Input Features

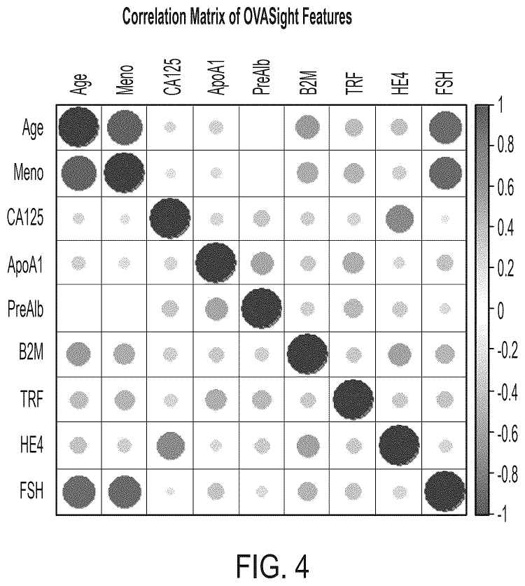

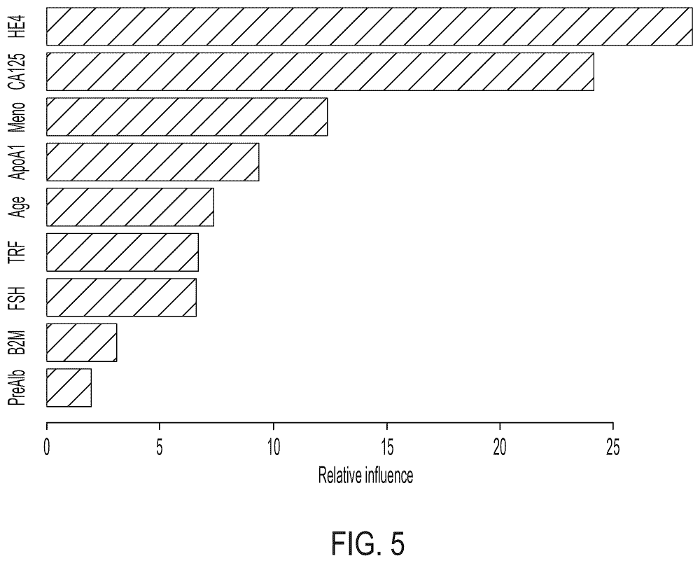

Seven biomarkers were used in the MIA3G algorithm: cancer antigen 125 (CA125), human epididymis protein 4 (HE4), beta-2 microglobulin (B2M), apolipoprotein A-1 (ApoA1), transferrin, transthyretin, and follicle stimulating hormone (FSH). CA125 and HE4 were chosen for their overexpression in many types of ovarian cancer[11-12]. The remaining biomarkers have demonstrated ability to detect malignancy in patients with low serum CA125 and/or HE4, such as early-stage malignancies, as well as reducing false positives in benign cases for which serum CA125 and/or HE4 were elevated for other reasons[13-16]. These features were examined for their correlation with each other and their contribution (FIGS. 4-5). Biomarker assays were performed using the Roche cobas 6000 analyzer, according to the manufacturer's instructions for use (Roche Corporation, Pleasonton, CA). In addition to these biomarkers, the patient's age and menopausal status were used as categorical input features. Menopause was defined as the absence of menses for >12 months.

### Example 4: Studies and Sample Sets

To create a highly generalizable classification algorithm, it was essential to train it on a diverse set of specimens with a wide reference range of biomarkers and other features. To this end, a heterogenous set of specimens was first created by combining samples from several prospective and retrospective IRB-approved studies (Table 2).

Broadly, the inclusion criteria for these studies were as follows:
Subjects age≥18 years
Informed consent provided by the subject
Subject was agreeable to phlebotomy
Subject had a documented pelvic mass which was planned for surgical intervention within 3 months of imaging. The pelvic mass was confirmed by imaging (computed tomography, ultrasonography, or magnetic resonance imaging) prior to enrollment.

Exclusion criteria included a diagnosis of malignancy in the previous 5 years (excepting nonmelanoma skin cancers). Exclusion criteria also included pelvic surgery within six weeks prior to enrollment in the study.

TABLE 2
Sample Set Composition

| Study | IRB/Protocol Number | N |
|---|---|---|
| OVAWatch Prospective Clinical | RP 08-2020, RP 05-2019, | 35 |
| Study [17] | RP 04-2019 | |
| Aspira Specimen (Serum) Bank | RP 01-2016/Pro00027159 | 290 |
| OVA1 Postmarket Study [18] | OVA1-PS1-CO4 | 1,385 |
| OVA500 Study [19] | OVA2-002-CO3 | 511 |
| University of Washington Study | OVA1-7788 | 218 |
| [20] | | |
| OVA1 Study [21] | OVA1-001-CO1 | 574 |
| BioBank [22] | SHARE v5.2 10 May 2021, | 54 |
| | IRB#: 2017-198 | |
| Total | | 3,067 |

This heterogenous set included a total of 3,067 samples (FIG. 1). The composite set was randomly broken into two non-overlapping sets such that:
1,067 samples were used for development of the algorithm and formed the training and testing set.
The remaining 2,000 samples were used for analytical validation.
Each set roughly received samples from every study proportionate to the size of the study.
The validation set had a prevalence rate of ˜5% (98 malignant and 1902 benign samples).

While the sample size and prevalence of malignancy were fixed, the sample assignment to each set was completely random, performed using a random number generator to remove any potential bias. The above binning of samples into development and validation sets ensured that not only was the assignment of samples fair and random, but it also allowed the algorithm to be trained/tested and then validated on sets that had an optimal level of similarities (and differences).

Table 3 details the clinicopathologic makeup of each set including age, pathology, histologic subtypes, and stages.

TABLE 3
Clinicopathologic breakdown of training, test, and validation datasets.

| | | Training Set | | | | | |
|---|---|---|---|---|---|---|---|
| Menopausal status | | All (N = 853) | | Pre (N = 410) | | Post (N = 443) | |
| Age (mean) | | 51.3 | | 40.3 | | 61.4 | |
| Pathology Diagnosis | | N | % | N | % | N | % |
| Benign ovarian conditions | | 548 | 64.20% | 319 | 77.80% | 229 | 51.70% |
| Low Malignant Potential | | 25 | 2.90% | 9 | 2.20% | 16 | 3.60% |
| (Borderline) | | | | | | | |
| Epithelial primary ovarian | | 200 | 23.40% | 49 | 12.00% | 151 | 34.10% |
| cancer | | | | | | | |
| Non-epithelial primary | | 41 | 4.80% | 19 | 4.60% | 22 | 5.00% |
| ovarian cancer | | | | | | | |
| Non-primary malignancies | | 39 | 4.60% | 14 | 3.40% | 25 | 5.60% |
| Stage | | | | | | | |
| (Primary Ovarian | | | | | | | |
| Malignancies) | | N | % | N | % | N | % |
| Stage I | | 90 | 37.30% | 30 | 44.10% | 60 | 34.70% |
| Stage II | | 33 | 13.70% | 10 | 14.70% | 23 | 13.30% |
| Stage III | | 83 | 34.40% | 16 | 23.50% | 67 | 38.70% |
| Stage IV | | 17 | 7.10% | 4 | 5.90% | 13 | 7.50% |
| Not Staged | | 18 | 7.50% | 8 | 11.80% | 10 | 5.80% |
| Histologic Subtype | | | | | | | |
| (Primary Ovarian | | | | | | | |
| Malignancies) | | N | % | N | % | N | % |
| Epithelial | Serous | 105 | 43.60% | 21 | 30.90% | 84 | 48.60% |
| Ovarian | Endometrioid | 31 | 12.90% | 10 | 14.70% | 21 | 12.10% |
| Cancer | Mucinous | 21 | 8.70% | 9 | 13.20% | 12 | 6.90% |
| (EOC) | Clear Cell | 18 | 7.50% | 4 | 5.90% | 14 | 8.10% |
| | Mixed | 12 | 5.00% | 2 | 2.90% | 10 | 5.80% |
| | Poorly | 6 | 2.50% | 1 | 1.50% | 5 | 2.90% |
| | Differentiated | | | | | | |
| | Transitional | 3 | 1.20% | 1 | 1.50% | 2 | 1.20% |
| | Cell | | | | | | |
| | Other | 4 | 1.70% | 1 | 1.50% | 3 | 1.70% |
| Non-EOC | Sex Cord | 20 | 8.30% | 10 | 14.70% | 10 | 5.80% |
| | Stromal | | | | | | |
| | Germ Cell | 11 | 4.60% | 8 | 11.80% | 3 | 1.70% |
| | Sarcoma/ | 9 | 3.70% | 0 | 0.00% | 9 | 5.20% |
| | Carcinosarcoma | | | | | | |
| | Other | 1 | 0.40% | 1 | 1.50% | 0 | 0.00% |
| | | Test Set | | | | | |
| Menopausal status | | All (N = 214) | | Pre (N = 105) | | Post (N = 109) | |
| Age (mean) | | 50.8 | | 40.5 | | 60.8 | |
| Pathology Diagnosis | | N | % | N | % | N | % |
| Benign ovarian conditions | | 152 | 71.00% | 86 | 81.90% | 66 | 60.60% |
| Low Malignant Potential | | 6 | 2.80% | 1 | 1.00% | 5 | 4.60% |
| (Borderline) | | | | | | | |
| Epithelial primary ovarian | | 45 | 21.00% | 12 | 11.40% | 33 | 30.30% |
| cancer | | | | | | | |
| Non-epithelial primary | | 5 | 2.30% | 3 | 2.90% | 2 | 1.80% |
| ovarian cancer | | | | | | | |
| Non-primary malignancies | | 6 | 2.80% | 3 | 2.90% | 3 | 2.80% |
| Stage | | | | | | | |
| (Primary Ovarian | | | | | | | |
| Malignancies) | | N | % | N | % | N | % |
| Stage I | | 15 | 30.00% | 6 | 40.00% | 9 | 25.70% |
| Stage II | | 5 | 10.00% | 1 | 6.70% | 4 | 11.40% |
| Stage III | | 24 | 48.00% | 7 | 46.70% | 17 | 48.60% |
| Stage IV | | 4 | 8.00% | 0 | 0.00% | 4 | 11.40% |
| Not Staged | | 2 | 4.00% | 1 | 6.70% | 1 | 2.90% |
| Histologic Subtype | | | | | | | |
| (Primary Ovarian | | | | | | | |
| Malignancies) | | N | % | N | % | N | % |
| Epithelial | Serous | 25 | 50.00% | 6 | 40.00% | 19 | 54.30% |
| Ovarian | Endometrioid | 5 | 10.00% | 4 | 26.70% | 1 | 2.90% |
| Cancer | Mucinous | 7 | 14.00% | 1 | 6.70% | 6 | 17.10% |
| (EOC) | Clear Cell | 3 | 6.00% | 1 | 6.70% | 2 | 5.70% |
| | Mixed | 4 | 8.00% | 0 | 0.00% | 4 | 11.40% |
| | Poorly | 1 | 2.00% | 0 | 0.00% | 1 | 2.90% |
| | Differentiated | | | | | | |
| | Transitional | 0 | 0.00% | 0 | 0.00% | 0 | 0.00% |
| | Cell | | | | | | |
| | Other | 0 | 0.00% | 0 | 0.00% | 0 | 0.00% |
| Non-EOC | Sex Cord | 1 | 2.00% | 1 | 6.70% | 0 | 0.00% |
| | Stromal | | | | | | |
| | Germ Cell | 0 | 0.00% | 0 | 0.00% | 0 | 0.00% |
| | Sarcoma/ | 4 | 8.00% | 2 | 13.30% | 2 | 5.70% |
| | Carcinosarcoma | | | | | | |
| | Other | 0 | 0.00% | 0 | 0.00% | 0 | 0.00% |
| | | Validation Set | | | | | |
| Menopausal status | | All (N = 2000) | | Pre (N = 1193) | | Post (N = 807) | |
| Age (mean) | | 47.5 | | 39.5 | | 59.4 | |
| Pathology Diagnosis | | N | % | N | % | N | % |
| Benign ovarian conditions | | 1836 | 91.80% | 1136 | 95.20% | 700 | 86.70% |
| Low Malignant Potential | | 66 | 3.30% | 31 | 2.60% | 35 | 4.30% |
| (Borderline) | | | | | | | |
| Epithelial primary ovarian | | 79 | 4.00% | 18 | 1.50% | 61 | 7.60% |
| cancer | | | | | | | |
| Non-epithelial primary | | 6 | 0.30% | 4 | 0.30% | 2 | 0.20% |
| ovarian cancer | | | | | | | |
| Non-primary malignancies | | 13 | 0.70% | 4 | 0.30% | 9 | 1.10% |
| Stage | | | | | | | |
| (Primary Ovarian | | | | | | | |
| Malignancies) | | N | % | N | % | N | % |
| Stage I | | 16 | 18.80% | 7 | 31.80% | 9 | 14.30% |
| Stage II | | 10 | 11.80% | 4 | 18.20% | 6 | 9.50% |
| Stage III | | 46 | 54.10% | 8 | 36.40% | 38 | 60.30% |
| Stage IV | | 5 | 5.90% | 0 | 0.00% | 5 | 7.90% |
| Not Staged | | 8 | 9.40% | 3 | 13.60% | 5 | 7.90% |
| Histologic Subtype | | | | | | | |
| (Primary Ovarian | | | | | | | |
| Malignancies) | | N | % | N | % | N | % |
| Epithelial | Serous | 46 | 54.10% | 7 | 31.80% | 39 | 61.90% |
| Ovarian | Endometrioid | 10 | 11.80% | 3 | 13.60% | 7 | 11.10% |
| Cancer | Mucinous | 6 | 7.10% | 2 | 9.10% | 4 | 6.30% |
| (EOC) | Clear Cell | 11 | 12.90% | 4 | 18.20% | 7 | 11.10% |
| | Mixed | 2 | 2.40% | 1 | 4.50% | 1 | 1.60% |
| | Poorly | 3 | 3.50% | 1 | 4.50% | 2 | 3.20% |
| | Differentiated | | | | | | |
| | Transitional | 0 | 0.00% | 0 | 0.00% | 0 | 0.00% |
| | Cell | | | | | | |
| | Other | 1 | 1.20% | 0 | 0.00% | 1 | 1.60% |
| Non-EOC | Sex Cord | 5 | 5.90% | 3 | 13.60% | 2 | 3.20% |
| | Stromal | | | | | | |
| | Germ Cell | 1 | 1.20% | 1 | 4.50% | 0 | 0.00% |
| | Sarcoma/ | 0 | 0.00% | 0 | 0.00% | 0 | 0.00% |
| | Carcinosarcoma | | | | | | |
| | Other | 0 | 0.00% | 0 | 0.00% | 0 | 0.00% |

### Training and Testing

The MIA3G algorithm was developed on 1,067 specimens comprised of proportionate samples from every study (FIG. 1) with 339 malignant and 728 benign samples resulting in a prevalence of 31%. This set was randomly divided a training set (n=853) and a non-overlapping testing set (n=214), representing 80% and 20% of the available samples, respectively. The algorithm was built on the training set and tested on the testing set to obtain an initial assessment of its performance. The performance metrics for the test data set are provided in Table 5.

The number of malignant and benign specimens were further balanced for algorithm training using an adaptation of the synthetic minority oversampling technique (SMOTE) that balances the minority and majority classes by creating synthetic observations near the decision boundary (called Borderline-SMOTE) [23]. The resulting dataset had an equivalent number of malignant and benign specimens, where the synthetic observations are close to the decision boundary. In the case of MIA3G the synthetic observations improved the algorithm's ability to discern between malignant and benign specimens. To improve malignancy detection a modestly higher weight was attached to the positive class during algorithm training in MIA3G. Weighing the malignant samples during training improved on the gains from balancing using the Borderline-SMOTE in positive detection, while having a negligible impact on benign discernment.

Several algorithms and software libraries were used to explore which technique would return the best risk classification for ovarian cancer. The caret library in R was used to screen 190 classification algorithms on the data [24]. Most algorithms in the caret library did not successfully classify ovarian cancer with a high level of sensitivity. Deep feed-forward neural networks demonstrated a high and balanced sensitivity, NPV and specificity leading to the selection of this algorithm for the development of MIA3G. Network hyperparameters evaluated during algorithm training and testing included: network architectures, activation functions, loss functions, node dropout for algorithm regularization, and learning rates. The final MIA3G algorithm was determined to be a network with these hyperparameters optimized to stratify malignancy risk. This algorithm was locked and used for subsequent analytical validation.

### Example 5: Analytical Performance Validation

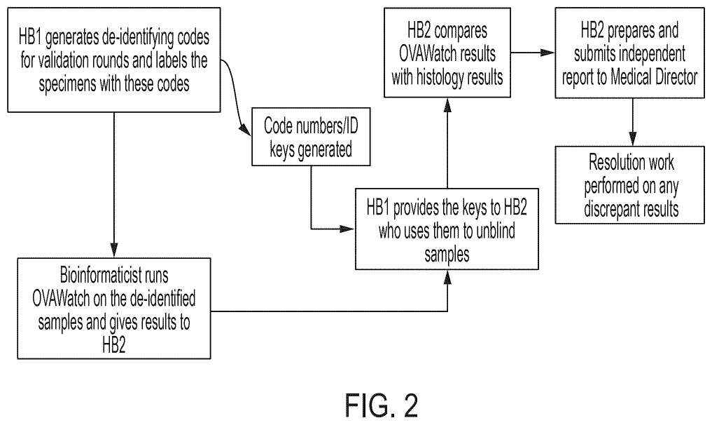

Analytical validation was performed on 2,000 samples with 98 malignant and 1,902 benign specimens resulting in a prevalence of 4.9%. Once the algorithm was developed and locked in a cloud-based HIPAA-compliant infrastructure, it was then run on the analytical validation samples in a blinded manner so that the person running the algorithm was blinded to the sample identities and their pathology results. Two honest brokers (HB1 and HB2) were employed to de-identify the samples, run the algorithm blinded, compare the classification of samples to the histology results and then issue an independent report containing performance metrics based on their findings (FIG. 2).

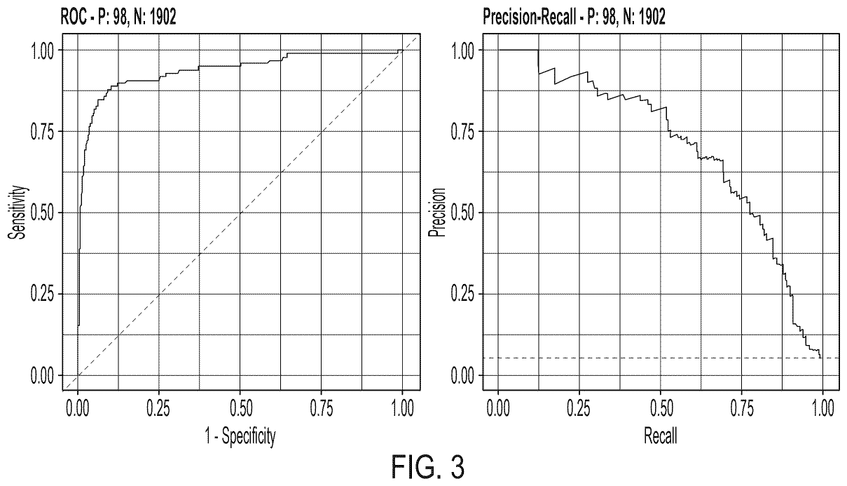

Performance metrics along with counts of true positives (TP), true negatives (TN), false positives (FP) and false negatives (FN) from analytical validation are provided in Table 4. Receiver operator characteristic (ROC) and Precision-Recall curves were also plotted (FIG. 3). Overall, a sensitivity of 89.8% and specificity of 84.02% were achieved, with an area under the curve (AUC) value of 0.938. MIA3G demonstrated an NPV of 99.38%. The PPV was lower at 22.45% due to the low prevalence of disease (˜5%) in this data set. Metrics have also been provided for specimens stratified by menopausal status, cancer stage, cancer type and malignancy potential. MIA3G was able to detect 20 out of 26 early-stage cancers (76.92% sensitivity) and misclassified only one late-stage malignancy (98.04% sensitivity). The algorithm also correctly classified 9 out of the 10 metastatic ovarian cancer cases (90% sensitivity) and 75 out of 79 instances of epithelial ovarian cancer (EOC), the most common type of ovarian cancer (94.94% sensitivity).

TABLE 4
Performance of MIA3G in the validation dataset. The number of cases or metrics not applicable
for that category are displayed by ‘—’. LMP indicates low malignant potential/borderline tumor.

| | Malignant | Benign | TP | TN | FP | FN | Sens | Spec | PPV | NPV |
|---|---|---|---|---|---|---|---|---|---|---|
| All | 98 | 1902 | 88 | 1598 | 304 | 10 | 89.80% | 84.02% | 22.45% | 99.38% |
| Pre-menopausal | 26 | 1167 | 21 | 1072 | 95 | 5 | 80.77% | 91.86% | 18.10% | 99.54% |
| Post-menopausal | 72 | 735 | 67 | 526 | 209 | 5 | 93.06% | 71.56% | 24.28% | 99.06% |
| EOC | 79 | — | 75 | — | — | 4 | 94.94% | — | — | — |
| Non-EOC | 6 | — | 1 | — | — | 5 | 16.67% | — | — | — |
| Stage I | 16 | — | 11 | — | — | 5 | 68.75% | — | — | — |
| Stage II | 10 | — | 9 | — | — | 1 | 90% | — | — | — |
| Stage III | 46 | — | 45 | — | — | 1 | 97.83% | — | — | — |
| Stage IV | 5 | — | 5 | — | — | 0 | 100% | — | — | — |
| Early Stage | 26 | — | 20 | — | — | 6 | 76.92% | — | — | — |
| (I & II) | | | | | | | | | | |
| Late Stage | 51 | — | 50 | — | — | 1 | 98.04% | — | — | — |
| (III & IV) | | | | | | | | | | |
| Not Staged | 8 | — | 6 | — | — | 2 | 75% | — | — | — |
| Non-Primary | 13 | — | 12 | — | — | 1 | 92.31% | — | — | — |
| LMP | — | 66 | — | 33 | 33 | — | — | 50.00% | — | — |
| Other Benigns | — | 1836 | — | 1565 | 271 | — | — | 85.24% | — | — |

After assessing several classification algorithms, MIA3G was trained on neural networks with the most balanced performance, and then tested on a heterogeneous cohort. The model was optimized to reduce overfitting and an oversampling technique was used to achieve a balanced performance which was higher than all other methods that were explored (Table 4). The training and testing stage used >1,050 specimens with >30% positive specimens indicative of a high-risk ovarian cancer population. This development was followed by a detailed validation process on 2,000 specimens that show performance in a low prevalence population (˜5%) making the algorithm highly generalizable. MIA3G has also been meticulously validated for its repeatability and reproducibility (Table 7).

The potential clinical utility of MIA3G in the evaluation of adnexal masses comes from its balanced performance which was facilitated by three development features: a large malignant set used in training and testing (N=339), the SMOTE technique applied to further boost the positive set, and a higher weight attached to the positive class. These features resulted in an algorithm with a high sensitivity, a vital feature given the high mortality of ovarian cancer, while retaining a high specificity. This high specificity drives a high negative predictive value in a population with a lower disease prevalence where clinical management options may include conservative management and at the same time minimizes the potentially lethal implications of false negatives in the context of cancer detection.

As a result of the random assignment of samples to the training and validation data sets, there was no way to match the distribution of cancer types between sets (Table 5). For example, by happenstance, five of the tumors in the validation set were stromal tumors and one was a germ cell tumor, subtypes known to have a different biomarker presentation compared to the more common epithelial types. In the test set, however, MIA3G demonstrated 100% sensitivity in non-epithelial malignancies, as sarcomas and carcinosarcomas comprised 4/5 non-epithelial malignancies in that set (Table 5). These cancer types present more similarly to EOC in terms of biomarker distribution. Nonepithelial subtypes are rare presentations of ovarian cancer, comprising approximately 10% of all ovarian malignancies [25], so their particularly low incidence presents a challenge with regard to generating sufficient data for training and validating machine learning algorithms. Future directions include evaluating how to train an algorithm on multiple subtypes that express different biomarker patterns and achieve consistent test performance across these subtypes.

The application of a deep neural network algorithm to biomarker testing opens significant areas for future study. Understanding where the algorithm “fails” provides an opportunity for deeper exploration into alternate biological explanations for false positive and false negative results. For example, there is a possibility that some combination of biomarkers may be identifying cancers outside of the ovaries, and therefore correctly suggesting malignancy, albeit not of ovarian origin. As a step for improvement, expanding the number and type of features that feed into the algorithm may help further enhance the sensitivity and specificity of the test. Preliminary efforts are underway to evaluate the addition of novel biomarkers and other modalities like microRNA, cell tumor DNA, and other genomic identifiers that may strengthen the algorithms' ability to both detect and rule out malignancy and advance the diagnostic ability of non-invasive testing.

The results described in Example 1 were obtained using the following materials and methods.

### Limiting Overfitting and Over-Sampling

Overfitting during model building was mitigated using randomized node dropout. Node dropout randomly drop units with their connections from the neural network during training. This prevents units from co-adapting too much, and excess weight being given to specific nodes. This significantly reduces overfitting and gives major improvements over other regularization methods[26].

BLS-SMOTE was also adopted for this purpose. Experiments conducted by Han et al. have shown that BLS-SMOTE approach achieves better TP rate and F-value than SMOTE and random over-sampling methods when working with imbalanced data. For every minority example, its k nearest neighbors from the same class are identified, then some examples are randomly selected from them according to the over-sampling rate[27]. After that, new synthetic examples are generated along the line between the minority example and its selected nearest neighbors. Unlike the existing over-sampling methods, BLS-SMOTE oversamples the borderline minority examples, which in many cases, in our cohort, were early-stage cancers.

### Feature Correlation

All the features were examined for any strong correlation in the context of the cohort by performing a correlation analysis between the features (FIG. 4). Quite understandably, age and menopausal status were highest correlated features the algorithm uses followed by FSH protein biomarker which was correlated to age and menopausal status. Removing menopausal status led to a modest decrease in algorithm performance metrics (n=10 different data randomizations, a mean decrease of 5.8% in sensitivity in the test data, specificity remained equivalent). While age and menopausal status were correlated it was deemed worth including both for the retention of sensitivity in algorithm performance shown in the test data set.

Similarly, removing FSH led to roughly equivalent sensitivity; however, there was a 3.5% decrease in specificity. Again, it was deemed worth including FSH for the retention of algorithm performance shown in the test data set. There were no other correlations in the data that were either ≥0.5 or ≤−0.5.

### Feature Contribution

Variable importance for each of the input features of the algorithm was also assessed (FIG. 5). Permutation-based variable importance analysis was used. As the permutations were stochastic, some variability was anticipated in the resulting importance depending on data seeding. FIG. 5 presents a representation of the mean 25 data randomization seeds. HE4, CA125, Menopausal status, and APOA1 age were the four most prominent features. This data along with the information from the correlation exploration suggested that all biomarkers and input variables were contributing in a meaningful manner to a variable extent.

### Test Set Performance

TABLE 5
Performance of MIA3G in the test dataset. The number of cases or metrics not applicable for
that category are displayed by ‘—’. LMP indicates low malignant potential/borderline tumor.

| | Malig | Benign | TP | TN | FP | FN | Sens | Spec | PPV | NPV |
|---|---|---|---|---|---|---|---|---|---|---|
| All | 56 | 158 | 51 | 139 | 19 | 5 | 91.07% | 87.97% | 72.86% | 96.53% |
| Pre-menopausal | 18 | 87 | 16 | 83 | 4 | 2 | 88.89% | 95.40% | 80.00% | 97.65% |
| Post-menopausal | 38 | 71 | 35 | 56 | 15 | 3 | 92.11% | 78.87% | 70.00% | 94.92% |
| EOC | 45 | — | 42 | — | — | 3 | 93.33% | — | — | — |
| Non-EOC | 5 | — | 5 | — | — | 0 | 100.00% | — | — | — |
| Stage I | 15 | — | 12 | — | — | 3 | 80.00% | — | — | — |
| Stage II | 5 | — | 5 | — | — | 0 | 100.00% | — | — | — |
| Stage III | 24 | — | 24 | — | — | 0 | 100.00% | — | — | — |
| Stage IV | 4 | — | 4 | — | — | 0 | 100.00% | — | — | — |
| Early Stage (I & II) | 20 | — | 17 | — | — | 3 | 85.00% | — | — | — |
| Late Stage (III & IV) | 28 | — | 28 | — | — | 0 | 100.00% | — | — | — |
| Not Staged | 2 | — | 2 | — | — | 0 | 100.00% | — | — | — |
| Not Primary to the Ovary | 6 | — | 4 | — | — | 2 | 66.67% | — | — | — |
| LMP | — | 6 | — | 3 | 3 | — | — | 50.00% | — | — |
| Other Benigns | — | 152 | — | 136 | 16 | — | — | 89.47% | — | — |

### Other Machine Learning Methods

Many other algorithms were also evaluated for their performance on the same dataset. Table 6 lists the performance of other machine learning algorithms included in this analysis. Neural Networks showed the highest sensitivity and negative predictive value (NPV) (the two metrics were optimized so as to minimize false negatives, a decision based on the high mortality of ovarian cancer, particularly when discovered at a late stage).

TABLE 6
Performance of other methods in comparison to Neural Networks
which demonstrated highest sensitivity and NPV.

| Model | Sens | Spec | PPV | NPV |
|---|---|---|---|---|
| C5.0 | 82.65 | 91.06 | 32.27 | 99.03 |
| Naïve Bayesian Classifier | 72.45 | 88.49 | 24.48 | 98.42 |
| Boosted Logistic Regression | 86.73 | 81.13 | 19.14 | 99.16 |
| SVM with Linear Kernel | 83.67 | 82.54 | 19.81 | 98.99 |
| Boosted Smoothing Spline | 79.59 | 86.54 | 23.35 | 98.8 |
| Generalized Linear Model | 83.67 | 83.39 | 20.6 | 99 |
| Self-Organizing Maps | 77.17 | 80.54 | 16.1 | 98.65 |
| Heteroscedastic Discriminatory Analysis | 59.18 | 98.26 | 63.74 | 97.9 |
| Neural Network | 89.80 | 84.02 | 22.45 | 99.38 |

### Precision

The MIA3G algorithm and individual analyte concentration measurements were rigorously evaluated for precision, i.e., repeatability and reproducibility according to CLSI standard EP05-A2 [28]. The precision study for MIA3G was designed to establish its performance across and within runs, days and operators. The exercise was configured to be run by two individual laboratory operators to assess the contribution of “between-operator” variability in MIA3G. Each sample was run in triplicate, at two separate times per day with a minimum of two hours apart to evaluate variability of MIA3G within and across runs (i.e., intra-reproducibility). Additionally, this process was repeated across 4 days to evaluate within and across day deviations (i.e., inter-reproducibility).

Repeatability and reproducibility of MIA3G probability risk scores were quantified in terms of percentage of coefficient of variation (% CV). % CV captures the extent of variability of data in relation to the mean of the population tested. It is the ratio of the standard deviation to the mean and is used for comparing the degree of variation from one data series to another, even if the means are drastically different from one another. A value of 10% CV or lower is a widely accepted degree of variability. Within experiment % CV captures repeatability and across experiment % CV demonstrates reproducibility (aka precision). MIA3G % CV are provided in Table 7 for three metrics: runs, days and operator. A low % CV (high repeatability and reproducibility) was demonstrated with all values being below or around 10% CV. Individual biomarkers also confirmed low variability at all three levels measured (data not shown).

TABLE 7
% CV Measurement of the MIA3G for Runs,
Days, and Operators by Sample (Pools)

| | Sample | | |
|---|---|---|---|
| | Serum Pool ID | | |
| MIA3G Risk Score | 25 | 26 | 27 |
| % CV within runs | 10.6 | 6.2 | 6.6 |
| % CV across runs | 0.0 | 0.0 | 0.9 |
| % CV within days | 10.7 | 6.3 | 6.8 |
| % CV across days | 0.0 | 3.0 | 3.6 |
| % CV within operator | 10.6 | 6.2 | 6.7 |
| % CV across operator | 0.0 | 0.0 | 0.0 |
| % CV overall error | 10.4 | 6.11 | 6.59 |

### Example 6: Performance of MIA3G in Retrospective Cases Independently Determined as Benign by Physicians

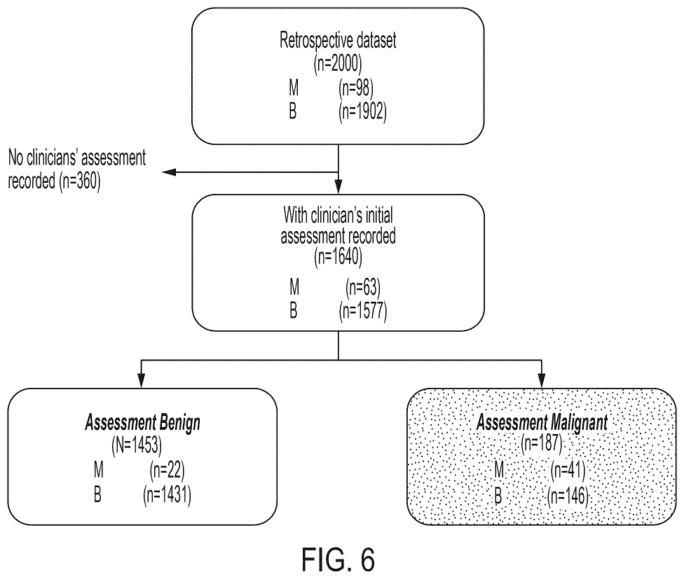

The deep neural network-based algorithm was clinically validated. The subset of patients that were assessment benign prior to surgery was analyzed to determine the performance of MIA3G in patients where the physician presumed the patient's mass to be benign. The workflow diagram for the derivation of retrospective data set of patients who were assessment benign (Multivariate Index Assay Benign “MIAB” data set) is presented in FIG. 6. It is important to note that all cases went to surgery and so had surgical pathology confirmation of diagnosis.

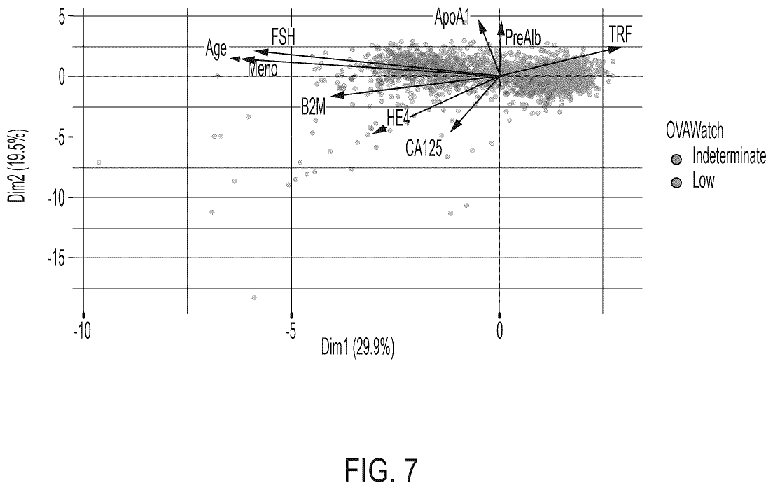

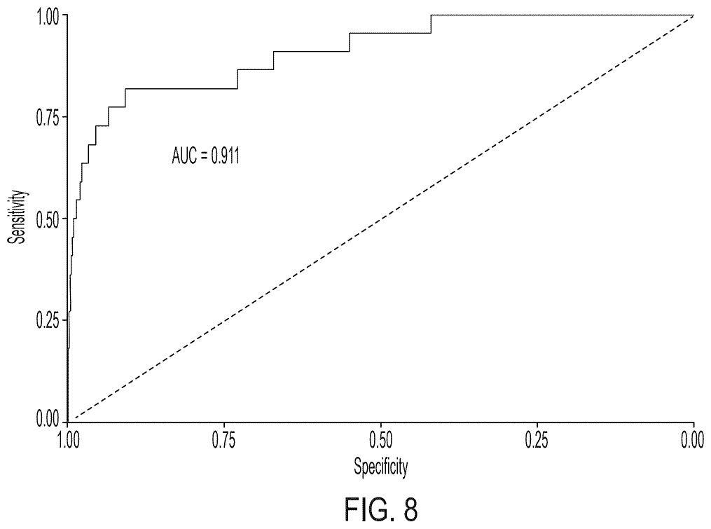

The influence of the seven biomarkers and clinical features (Age and menopausal status) that contribute to classification of low probability of malignancy or indeterminate risk are summarized in the plot of the principal components analysis of the entire 2000 sample validation set (FIG. 7). This analysis shows that CA125 and HE4 are positively correlated with classification of indeterminate, whereas TRF and PreAlb are positively correlated with the classification of low probability of malignancy. In the MIAB data set, which comprised 1453 of 2000 validation samples, the prevalence was 1.5% (22/1453). The performance of MIA3G in the MIAB data set is shown graphically in the Receiver Operator Characteristics (ROC) plot in FIG. 8 and is presented for the threshold MIA3G score of ≥5.0 as indeterminate in Table 8.

TABLE 8
Performance of MIA3G in identifying histologically
malignant patients in the MIAB data set at
an MIA3G score threshold value of ≥5.0

| | | | | Positive | Negative |
|---|---|---|---|---|---|
| | | | | Predictive | Predictive |
| | | Sensitivity | Specificity | Value | Value |
| All | Estimate | 81.8 | 87.4 | 9.1 | 99.7 |
| (N = 1453) | (%) | | | | |
| | n/N | 18/22 | 1251/1431 | 18/198 | 1251/1255 |
| | 95% CI | 61.5-92.7 | 85.6-89.0 | 5.8-13.9 | 99.2-99.9 |

MIA3G at a threshold of ≥5.0 had a sensitivity of 81.8.% (95% CI: 61.5-92.7) a specificity of 87.4% (95% CI: 85.6-89.0) and an NPV of 99.7% (99.2-99.9) for detecting histologically malignant patients in this group, as compared to the sensitivity of 89.8% and specificity of 84.0% and a negative predictive value (NPV) of 99.4% in all evaluated patients, presented previously [Example 1]. MIA3G identified 18 of 22 patients as indeterminate that were not determined as assessment benign by physician assessment alone. Conversely, in patients who were assessment malignant, (187 of 1640 samples), MIA3G identified all histologically malignant cases as indeterminate (41/41). MIA3G had a higher rate of false positives than physician assessment. In assessment benign patients, MIA3G identified as indeterminate 180 patients who were histologically benign, or a false positive rate of 92.4%.

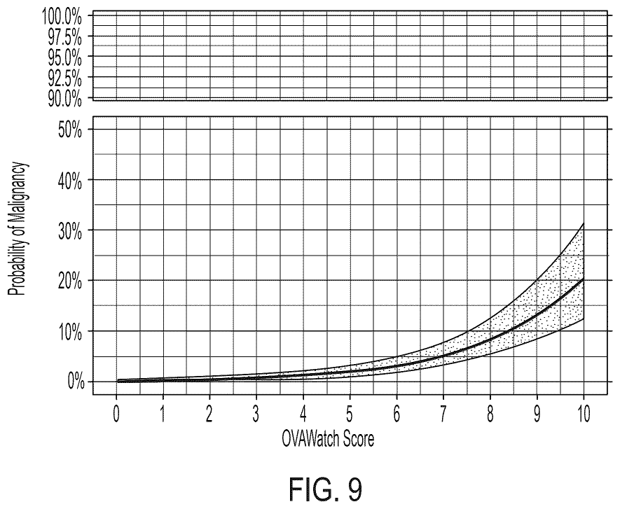

The probability of a malignant mass by MIA3G score in the MIAB data set is shown in FIG. 9. At this low prevalence, this probability is below 5% at the threshold MIA3G score of 5.0. Table 9 shows the characteristics of histologically malignant patients in the assessment benign group. The malignancies that MIA3G called low probability of malignancy are: MIAB 7, MIAB 9, MIAB 12 and MIAB 19.

TABLE 9
Characteristics of all patients in the MIAB dataset who were histologically
malignant. Shaded rows are false negative cases

| | | Menopausal | | Histological | | MIA3G |
|---|---|---|---|---|---|---|
| Subject | Age | status | Classification | Subtype | Stage | Score |
| MIAB 1 | 56 | Postmenopausal | EOC | Serous | III | 9.5 |
| MIAB 2 | 57 | Postmenopausal | EOC | Serous | III | 9.5 |
| MIAB 3 | 47 | Premenopausal | EOC | Clear Cell | III | 9.5 |
| MIAB 4 | 53 | Postmenopausal | EOC | Clear Cell | I | 8.5 |
| MIAB 5 | 40 | Premenopausal | EOC | Clear Cell | I | 9 |
| MIAB 6 | 82 | Postmenopausal | Metatstatic | NA | NA | 9.5 |
| MIAB 7 | 35 | Premenopausal | EOC | Mucinous | I | 1.5 |
| MIAB 8 | 58 | Premenopausal | Metatstatic | NA | NA | 8 |
| MIAB 9 | 68 | Postmenopausal | EOC | NA | NA | 1 |
| MIAB | 72 | Postmenopausal | EOC | Serous | I | 9.5 |
| 10 | | | | | | |
| MIAB | 70 | Postmenopausal | EOC | Mucinous | I | 9.5 |
| 11 | | | | | | |
| MIAB | 49 | Premenopausal | EOC | Granulosa Cell | I | 0 |
| 12 | | | | Tumor | | |
| MIAB | 41 | Premenopausal | EOC | Endometrioid | NS | 9.5 |
| 13 | | | | | | |
| MIAB | 52 | Postmenopausal | EOC | Endometrioid | I | 9.5 |
| 14 | | | | | | |
| MIAB | 49 | Premenopausal | EOC | Endometrioid | II | 9.5 |
| 15 | | | | | | |
| MIAB | 71 | Postmenopausal | EOC | Serous | II | 6.5 |
| 16 | | | | | | |
| MIAB | 58 | Postmenopausal | EOC | Clear Cell | NS | 9 |
| 17 | | | | | | |
| MIAB | 58 | Postmenopausal | EOC | Serous | III | 9.5 |
| 18 | | | | | | |
| MIAB | 58 | Postmenopausal | EOC | Serous | NS | 0.5 |
| 19 | | | | | | |
| MIAB | 62 | Postmenopausal | Metatstatic | NA | NA | 9.5 |
| 20 | | | | | | |
| MIAB | 35 | Premenopausal | EOC | Adenocarcinoma of | NA | 9.5 |
| 21 | | | | colon, metastatic to | | |
| | | | | ovaries | | |
| MIAB | 42 | Premenopausal | Metatstatic | NA | NA | 9.5 |
| 22 | | | | | | |

### Example 7: Performance of MIA3G in a Prospective Low-Prevalence Study

Validation data was collected in a prospective clinical study of intended-use patients (Table 10), and analyzed retrospectively.

TABLE 10
Enrollment sites and internal and Institutional Review Board
(IRB) study identifiers included in the prospective analyses.

| | En- | First Patient | |
|---|---|---|---|
| Site Name/Number | rollment | Enrolled | Study/IRB# |
| Axia Women's Health/01 | 50 | Jun. 25, 2020 | OVANex/08- |
| | | | 2020 |
| May Grant OBGYN/04 | 110 | Oct. 21, 2020 | OVANex/08- |
| | | | 2020 |
| Hill Country OBGYN/05 | 94 | Jan. 21, 2021 | OVANex/08- |
| | | | 2020 |
| Square Medical | 68 | Dec. 8, 2020 | OVANex/08- |
| OBGYN/06 | | | 2020 |
| Premier OBGYN/09 | 44 | Mar. 19, 2021 | OVANex/08- |
| | | | 2020 |
| MidTown OBGYN/10 | 33 | Sep. 23, 2021 | OVANex/08- |
| | | | 2020 |
| Women's Health of | 6 | Mar. 23, 2022 | OVANex/08- |
| Mobile/11 | | | 2020 |
| New Horizon's Clinical | 4 | May 17, 2022 | OVANex/08- |
| Trials/12 | | | 2020 |
| Altus Research/03 | 130 | Sep. 21, 2020 | OVANex/04- |
| | | | 2019 |
| Northwell Health/08 | 82 | Jul. 13, 2021 | OVANex/04- |
| | | | 2019 |
| New Horizon's Clinical | 96 | Jan. 19, 2021 | OVANex/05- |
| Trials/04 | | | 2020 |

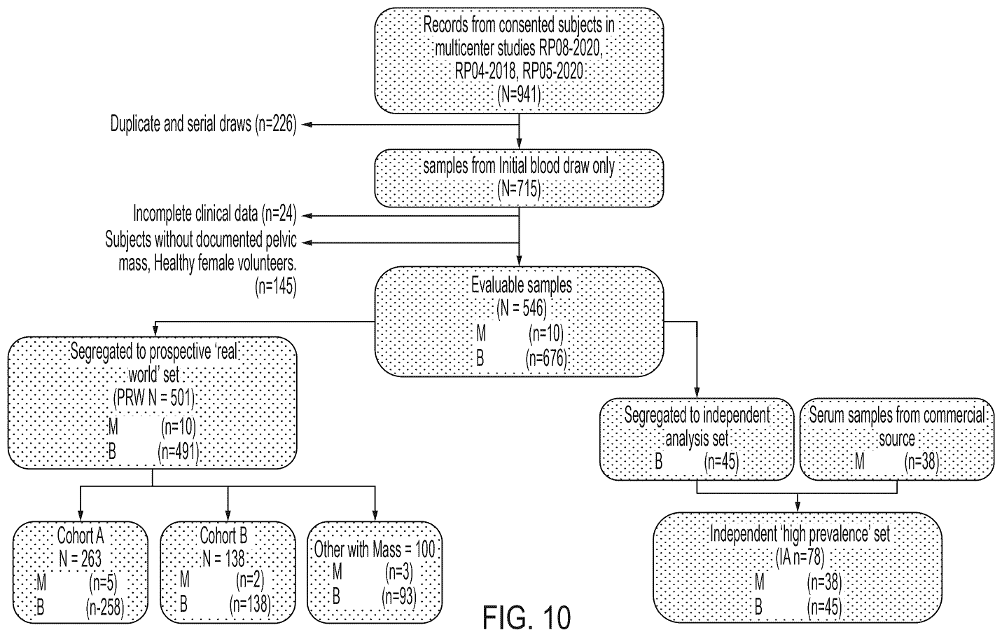

Physicians did not have access to MIA3G to support clinical decisions. Some patients received multiple blood draws and tests at suggested intervals throughout the study as part of the protocol, but these exact intervals were determined by the physicians. For this analysis, data was only included from the patient's initial blood draw and tests. The first draw was focused on only because it would allow the most direct assessment of the test's sensitivity to detecting malignancy. The flow diagram describing how the data set was comprised is shown in FIG. 10. Of 546 evaluable patients in this data set, 133 had surgery for their masses. The prospective data was further divided into a low-prevalence prospective real world (PRW) validation set and an independent analysis set (IA). The composition of the samples distributed into the data sets is summarized in Table 11.

TABLE 11
Prospective study patient demographic and clinical characteristics.

| N | | All | | Premenopausal | | Postmenopausal | |
|---|---|---|---|---|---|---|---|
| Individual Patients | | 546 | | 344 | | 202 | |
| Mean Age | | 47.5 | | 41.0 | | 58.7 | |
| Race and/or Ethnicity | | N | % | N | % | N | % |
| White/Caucasian | | 339 | 62.1% | 195 | 56.7% | 144 | 71.3% |
| Black or African American | | 44 | 8.1% | 26 | 7.6% | 18 | 8.9% |
| Asian | | 22 | 4.0% | 20 | 5.8% | 2 | 1.0% |
| Hispanic or Latino | | 19 | 3.5% | 17 | 4.9% | 2 | 1.0% |
| Ashkenazi Jewish | | 1 | 0.2% | 0 | 0.0% | 1 | 0.5% |
| Indigenous American or Alaska | | 2 | 0.4% | 2 | 0.6% | 0 | 0.0% |
| Native | | | | | | | |
| Native Hawaiian or other Pacific | | 2 | 0.4% | 1 | 0.3% | 1 | 0.5% |
| Islander | | | | | | | |
| Other or more than one of the above | | 76 | 13.9% | 53 | 15.4% | 23 | 11.4% |
| Unknown | | 41 | 7.5% | 30 | 8.7% | 11 | 5.4% |
| Non-surgery patients, presumed | | 395 | 72.3% | 261 | 75.9% | 134 | 66.3% |
| benign (n, %) | | | | | | | |
| Patients with surgical pathology | | 151 | 27.7% | 83 | 24.1% | 68 | 33.7% |
| (n, %) | | | | | | | |
| Pathology Diagnosis | | N | % | N | % | N | % |
| Benign ovarian conditions | | 140 | 92.7% | 77 | 92.8% | 63 | 92.6% |
| Low Malignant Potential | | 1 | 0.7% | 1 | 1.2% | 0 | 0.0% |
| (Borderline) | | | | | | | |
| Epithelial ovarian cancer | | 5 | 3.3% | 2 | 2.4% | 3 | 4.4% |
| Non-epithelial primary ovarian | | 4 | 2.6% | 2 | 2.4% | 2 | 2.9% |
| cancer | | | | | | | |
| Non-primary malignancies | | 1 | 0.7% | 1 | 1.2% | 0 | 0.0% |
| Stage (Primary Ovarian Malignancies) | | N | % | N | % | N | % |
| Stage I | | 3 | 33.3% | 1 | 25.0% | 2 | 40.0% |
| Stage II | | 2 | 22.2% | 2 | 50.0% | 0 | 0.0% |
| Not Staged | | 3 | 33.3% | 0 | 0.0% | 3 | 60.0% |
| Histologic Subtype (Primary Ovarian | | | | | | | |
| Malignancies) | | N | % | N | % | N | % |
| Epithelial | Serous | 1 | 11.1% | 0 | 0.0% | 1 | 20.0% |
| Ovarian | Endometrioid | 1 | 11.1% | 0 | 0.0% | 1 | 20.0% |
| | Mixed | 1 | 11.1% | 1 | 25.0% | 0 | 0.0% |
| Non-EOC | Sex Cord Stromal (Granulosa | 2 | 22.2% | 0 | 0.0% | 2 | 40.0% |
| | Cell Tumor) | | | | | | |
| | Sex Cord Stromal (Sertoli- | 1 | 11.1% | 1 | 25.0% | 0 | 0.0% |
| | Leydig Tumor) | | | | | | |
| | Carcinosarcoma | 1 | 11.1% | 0 | 0.0% | 1 | 20.0% |
| | Leiomyosarcoma | 1 | 11.1% | 1 | 25.0% | 0 | 0.0% |

The PRW data set had a prevalence of 9.400 (10/106) when considering only histologically confirmed malignancies, and 2.0% (10/501) when considering all first-draw patients. One patient had a confirmed Low Malignant Potential (Borderline) tumor that was considered benign for this analysis.

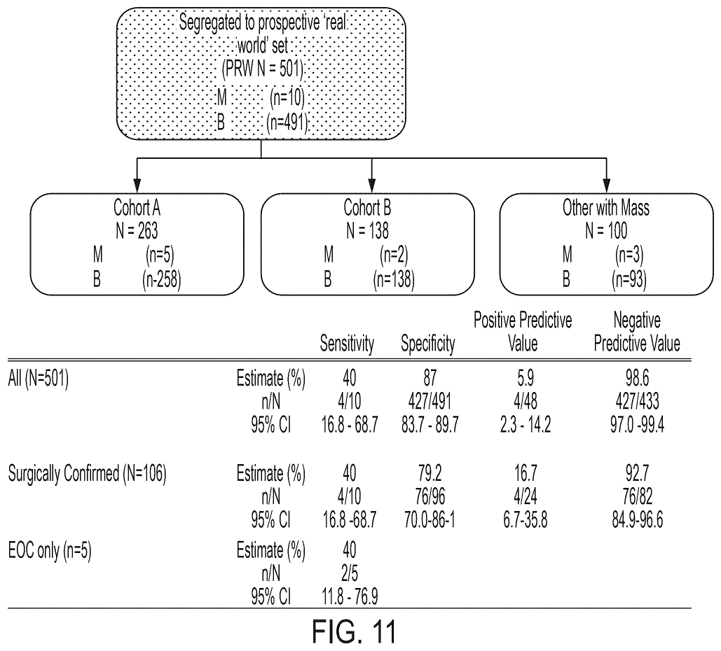

To examine the performance of MIA3G in a real-world setting, evaluable patients that did not go to surgery were considered as histologically benign in these analyses because they have been followed at least 5 months with TVUS without a reported significant increase in size. This was to approximate the tests' clinical utility by integrating independent physician assessment into the overall risk assessment. MIA3G, at a previously validated threshold value of ≥5.0 [Example 1] identified 4 of 10 (sensitivity of 40%) histologically malignant patients as indeterminate (Table 9). Of the 10 total histologically confirmed malignancies, 50% (5) were not epithelial ovarian cancers (EOC), and 50% were considered early-stage (FIG. 11). This contrasts with the distribution in a previous analytical validation where EOC represented 80.6% (79/98) malignancies and non-EOC malignancies were 7.6% (6/79) of all malignancies [Example 1]. Early-stage cancers comprised 26 of the 79 malignancies. The false positive rate was 12.8% (64/501) when including patients who did not go to surgery, compared with 15.2% (304/2000) in the published validation report [Example 1].

The study protocols had physicians stratify patients into cohorts based on whether the patient showed physical symptoms (eg. pain, bloating, unexplained weight loss, frequent urination) and imaging (TVUS or CT) confirmation of an adnexal mass (Cohort A) or showed no physical symptoms but a mass was present by imaging. For this analysis, cases from Cohort C were lumped in with the cases where the cohort was not indicated by the physician into a single “Other, with Mass” cohort. Table 12 summarizes the performance of MIA3G at a score threshold of >5.0 in the cohorts for identifying histologically malignant patients as indeterminate. There were differences in sensitivity among these cohorts, but small sample sizes warrant against any comparisons. NPV was above 98% for these cohorts.

TABLE 12
Performance of MIA3G stratified by symptom cohorts

| | | | | Positive | Negative |
|---|---|---|---|---|---|
| | | | | Predictive | Predictive |
| | | Sensitivity | Specificity | Value | Value |
| Cohort A | Estimate (%) | 60 | 88.4 | 9.1 | 99.1 |
| (N = 263) | n/N | 3/5 | 228/258 | 3/33 | 228/230 |
| | 95% CI | 23.1-88.2 | 83.9-91.2 | 3.1-23.6 | 96.9-99.8 |
| Cohort B | Estimate (%) | 0.0 | 89.0 | 0.0 | 98.4 |
| (N = 138) | n/N | 0/2 | 121/136 | 0/15 | 121/123 |
| | 95% CI | 0.0-65.8 | 82.6-93.2 | 0.0-20.4 | 94.3-99.6 |
| Other | Estimate (%) | 33.3 | 80.4 | 5.0 | 98.4 |
| With Mass | n/N | 1/3 | 78/97 | 1/19 | 78/80 |
| (N = 100) | 95% CI | 6.1-79.2 | 71.4-87.1 | 9.0-23.6 | 91.3-99.3 |

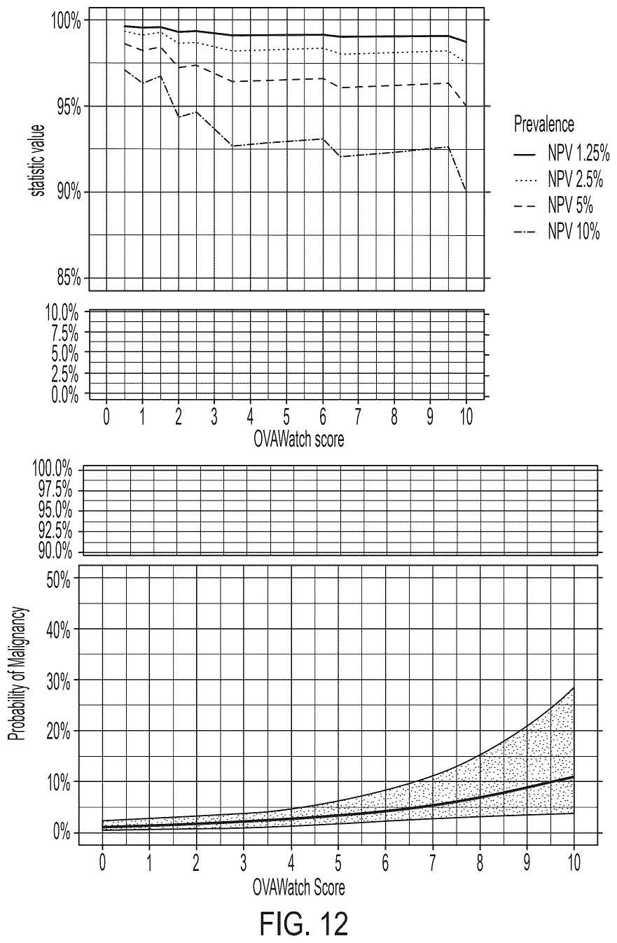

The performance characteristics of MIA3G were analyzed across the range of threshold scores to evaluate the stability of performance as a function of thresholds and prevalence. The NPV and positive predictive value (PPV) were calculated for estimated prevalence between 1.25 to 1000 using the formulae presented in the Methods. The results are presented graphically in FIG. 12. As expected, specificity and PPV increased as MIA3G scores increased, and sensitivity and NPV increased as scores decreased. NPV remained stable across the spectrum of MIA3G scores. Comparable performance values at projected prevalence of 1.25, 2.5%, 5.0% and 10.000 are also shown in Table 13.

TABLE 13
Sensitivity, specificity, negative predictive value (NPV) and positive predictive value (PPV)
estimated from prevalence as a function of OVAWatch score in data from the PRW dataset.

| MIA3G | | | 1.25% | | 2.50% | | 5% | | 10% | |
|---|---|---|---|---|---|---|---|---|---|---|
| Score | Sensitivity | Specificity | NPV | PPV | NPV | PPV | NPV | PPV | NPV | PPV |
| 0 | 100.0% | 0.0% | NA | 1.3% | NA | 2.5% | NA | 5.0% | NA | 10.0% |
| 0.5 | 90.0% | 37.7% | 99.7% | 1.8% | 99.3% | 3.6% | 98.6% | 7.1% | 97.1% | 13.8% |
| 1 | 80.0% | 58.0% | 99.6% | 2.4% | 99.1% | 4.7% | 98.2% | 9.1% | 96.3% | 17.5% |
| 1.5 | 80.0% | 66.6% | 99.6% | 2.9% | 99.2% | 5.8% | 98.4% | 11.2% | 96.8% | 21.0% |
| 2 | 60.0% | 74.5% | 99.3% | 2.9% | 98.6% | 5.7% | 97.3% | 11.0% | 94.4% | 20.8% |
| 2.5 | 60.0% | 78.8% | 99.4% | 3.5% | 98.7% | 6.8% | 97.4% | 13.0% | 94.7% | 23.9% |
| 3 | 50.0% | 81.9% | 99.2% | 3.4% | 98.5% | 6.6% | 96.9% | 12.7% | 93.6% | 23.5% |
| 3.5 | 40.0% | 83.7% | 99.1% | 3.0% | 98.2% | 5.9% | 96.4% | 11.4% | 92.6% | 21.4% |
| 3.5 | 40.0% | 85.3% | 99.1% | 3.3% | 98.2% | 6.5% | 96.4% | 12.6% | 92.8% | 23.3% |
| 4.5 | 40.0% | 86.2% | 99.1% | 3.5% | 98.2% | 6.9% | 96.5% | 13.2% | 92.8% | 24.3% |
| 4.5 | 40.0% | 87.0% | 99.1% | 3.7% | 98.3% | 7.3% | 96.5% | 13.9% | 92.9% | 25.4% |
| 5 | 40.0% | 88.2% | 99.1% | 4.1% | 98.3% | 8.0% | 96.5% | 15.1% | 93.0% | 27.3% |
| 6 | 40.0% | 89.6% | 99.2% | 4.6% | 98.3% | 9.0% | 96.6% | 16.9% | 93.1% | 30.0% |
| 6.5 | 30.0% | 90.6% | 99.0% | 3.9% | 98.1% | 7.6% | 96.1% | 14.4% | 92.1% | 26.2% |
| 7 | 30.0% | 91.0% | 99.0% | 4.1% | 98.1% | 7.9% | 96.1% | 15.0% | 92.1% | 27.1% |
| 7.5 | 30.0% | 92.1% | 99.0% | 4.6% | 98.1% | 8.8% | 96.2% | 16.6% | 92.2% | 29.6% |
| 8 | 30.0% | 93.1% | 99.1% | 5.2% | 98.1% | 10.0% | 96.2% | 18.6% | 92.3% | 32.5% |
| 8.5 | 30.0% | 94.5% | 99.1% | 6.5% | 98.1% | 12.3% | 96.2% | 22.3% | 92.4% | 37.7% |
| 9 | 30.0% | 95.5% | 99.1% | 7.8% | 98.2% | 14.7% | 96.3% | 26.1% | 92.5% | 42.7% |
| 9.5 | 30.0% | 98.2% | 99.1% | 17.2% | 98.2% | 29.6% | 96.4% | 46.3% | 92.7% | 64.5% |
| 10 | 0.0% | 100.0% | 98.8% | NA | 97.5% | NA | 95.0% | NA | 90.0% | NA |

Additional details regarding the surgical pathology-identified malignancies are presented in Table 14, to further understand the factors contributing to the misclassification of the malignancies. Misclassification of the malignant cases by MIA3G was not associated with any of the features presented in the Table.

TABLE 14
MIA3G score in PRW patients with malignancies. The
cases that are misclassified as low risk by MIA3G
score are: PRW2, PRW3, PRW6, PRW7, PRW9 and PRW10.

| | Study | Menopausal | | | Histological | | MIA3G |
|---|---|---|---|---|---|---|---|
| Subject | Cohort | Status | Age | Type | Subtype | Stage | Score |
| PRW1 | A | Premenopausal | 53 | Other | Mucinous, | NS* | 9.5 |
| | | | | | intestinal origin | | |
| PRW2 | A | Premenopausal | 42 | Other | Leiomyosarcoma | NS | 0.5 |
| PRW3 | A | Premenopausal | 45 | EOC | Endometrioid | NS | 1.5 |
| PRW4 | A | Premenopausal | 55 | EOC | Mixed | II | 9.5 |
| PRW5 | A | Postmenopausal | 53 | Other | Granulosa Cell | II | 6.0 |
| PRW6 | B | Postmenopausal | 80 | EOC | Epithelial | NS | 2.5 |
| | | | | | Carcinosarcoma | | |
| PRW7 | B | Postmenopausal | 54 | Other | Granulosa Cell | NS | 1.5 |
| PRW8 | Other | Postmenopausal | 45 | EOC | Endometrioid | NS | 9.5 |
| PRW9 | Other | Postmenopausal | 65 | EOC | Serous | I | 3.0 |
| PRW10 | Other | Premenopausal | 21 | Other | Sertoli-Leydig | I | 0.0 |

*NS—Not Staged
NS = Not Staged

### Example 8: Performance of MIA3G in an Independent High Prevalence Data Set

The MIA3G performance in a high-prevalence population was addressed by assembling a data set of independent prospective specimens and specimens of known pathology obtained from commercial sources. This was because NPV and PPV are prevalence-dependent, and it was necessary to evaluate how the test might perform in a clinical context where prevalence is variable.

The performance of the independent validation cohort is summarized in Table 15. This cohort was selected from early clinical trial results and supplemented with serum samples from patients with surgical pathology-confirmed malignancies with the goal of producing a simulated high prevalence data set. The prevalence in this data set was 45.8% (38/83) In this high-prevalence cohort (41%), MIA3G showed 83.3% sensitivity (95% CI: 69.6%-92.6%) and 90.2% (CI of 85.2% to 98.8%) specificity over all samples. The NPV was 87.8% (95% CI:75.8%-94.3%).

TABLE 15
Performance of MIA3G in a high-prevalence independent data set.

| | | | | Positive | Negative |
|---|---|---|---|---|---|
| | | Sensi- | Speci- | Predictive | Predictive |
| | | tivity | ficity | Value | Value |
| All | Estimate (%) | 84.2 | 95.6 | 94.1 | 87.8 |
| (N = 83) | n/N | 32/38 | 43/45 | 34/34 | 43/49 |
| | 95% CI | 69.6-92.6 | 85.2-98.8 | 80.9-98.4 | 75.8-94.3 |

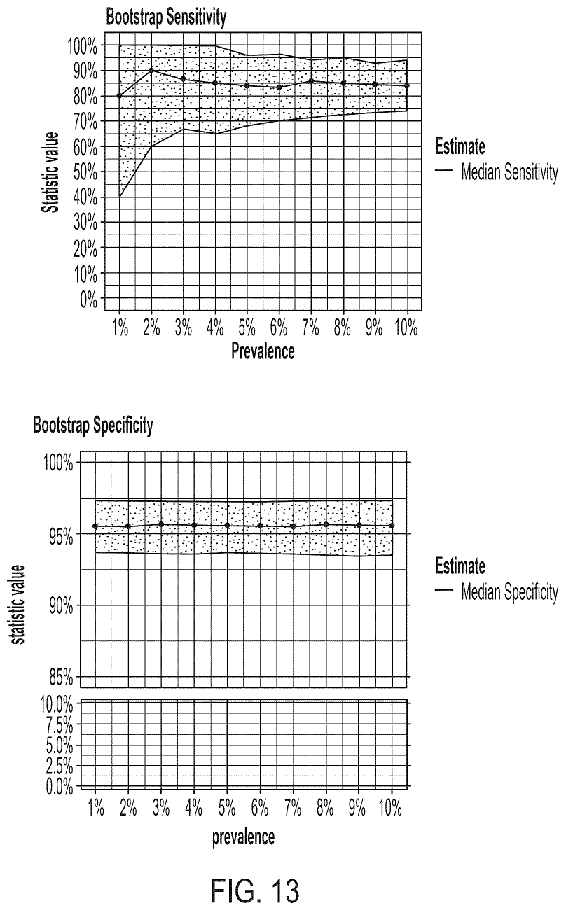

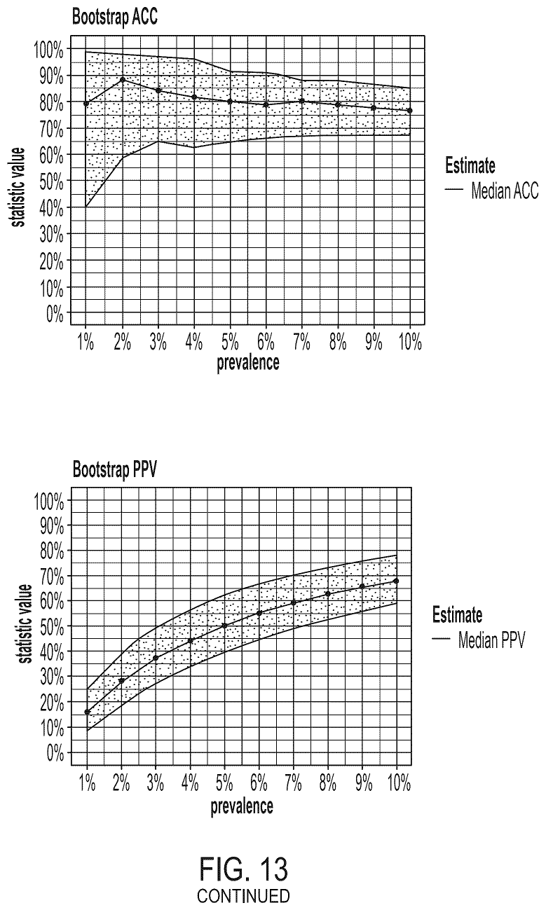

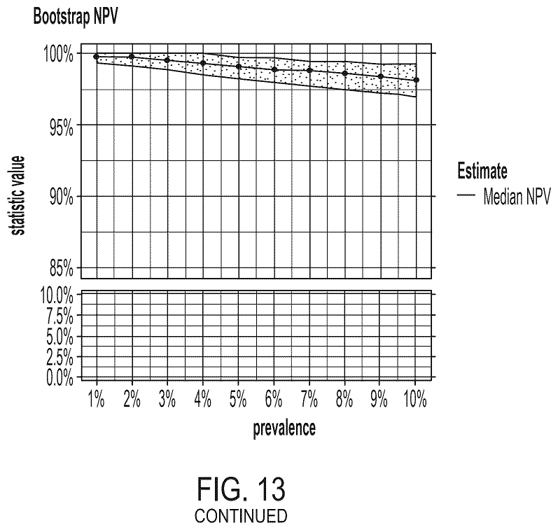

The influence of prevalence on MIA3G performance from this data set was also examined using bootstrap analysis, simulating sets with increasing prevalence (approximately 1-10%) but similar in size to the PRW data set (N=501). The results are presented in FIG. 13. These simulations showed that sensitivity and PPV were most susceptible to prevalence effects. PPV varied in magnitude over prevalence, as expected. Sensitivity showed consistent median level but much larger variance at lower prevalence. NPV and specificity did not vary more than 10% in magnitude over this range.

Many adnexal masses discovered on initial clinical examination can be managed conservatively due to intrinsically low risk of malignancy [29-31]. Unnecessary surgical intervention can result in possible surgical complications, loss of productivity, and increased costs to patients [44-45]. Although several tools exist to assess the need for surgical management of adnexal masses suspected to be malignant [38,39,46], there have not been effective biomarker-based tests to aid the clinical management of women with adnexal mass that is suspected to be benign, and thus nothing to specifically guide conservative management of a mass.

The proposed intended use of MIA3G is as a non-invasive test to assess the risk of ovarian cancer for women with adnexal masses evaluated by initial clinical assessment as indeterminate or benign. An effective biomarker-based test would need to have the following properties: 1) a high NPV for ruling out malignancy when the result is low risk, which would be most of the cases in this intended use group 2) a good sensitivity to not miss a possible malignancy that physicians would otherwise miss using other assessment methods 3) reasonable specificity so as not to place benign masses into a high-risk category. The results presented here supports the fact that MIA3G achieves these design goals when properly integrated with current clinical practice.

In the PRW sample set, MIA3G at a threshold score of 5.0 had a NPV of 92.7% for surgically confirmed samples and 98.6% for all samples; these values are within the limits of previous studies [Example 1]. NPV was consistent across the cohorts. This validates a role for this test in confirming a benign. The sensitivity of the test (40%, 95% CI: 16.8%-68.7%) was much lower in the PRW data set than in the retrospective validation report [Example 1] and the MIAB data set presented here (sensitivity of 81.8%, (95% CI 61.5%-92.7%), though the difference was not statistically significant due broad overlapping confidence intervals. This lower sensitivity needs to be acknowledged because it is contrary to the application as a Rule-Out test and was generally poor at the 5.0 threshold value across the cohorts. Our bootstrap investigations suggest that at this low prevalence, however, low sensitivity estimates can be a result of sampling, even where the true population sensitivity may be high. NPV however was more stable across the prevalence range in the bootstrap study and will be high in most low-prevalence situations due to the nature of the calculation. This favors a rule-out test where the expected prevalence of malignancy in women presenting with a mass is likely less than 10%. Simple adnexal masses have very low risks of malignancy (0-1%) and in masses that are indeterminate by ultrasonography, the incidence is less than 5% [32,33, 47].

Another contribution to low sensitivity in the PRW may be from the distribution of types and stages of malignancies. Several unusual malignancies were discovered in the PRW study; two Sertoli-Leydig (SLCT) tumors, two granulosa cell tumors (GCT), and one presumed uterine leiomyosarcoma. Clinical data revealed the leiomyosarcoma was diagnosed on a true cut biopsy of a pelvic mass. This most likely was the uterus. At ultrasound examination, most GCTs are large multilocular-solid masses or solid tumors. Tumor markers with this clinical presentation would include inhibin levels, Antimillerian hormone, or Müllerian-inhibiting substance [48]. Sertoli-Leydig cell tumors makeup <0.5% of all ovarian tumors and are benign or malignant, androgen-secreting tumors. They are unilateral and contain solid elements. Patients with Sertoli-Leydig cell tumors often present with masculinization. Testosterone and estrogen levels are appropriate markers [49]. These rare tumor types should be suspected on clinical grounds and appropriate tumor markers drawn. Additionally, and as expected for benign mass management data set, a higher percentage of the masses were early stage (50%) as compared to the validation set [Example 1] and published studies of higher risk patients [38,39]. Serial monitoring may increase the frequency of early detection in these patients [50].

The role of the physician in the initial triage of adnexal mass was not systematically investigated in these prospective studies but is likely to play a role in overall diagnostic accuracy for adnexal mass risk of malignancy. Information was not collected on clinical covariates that influenced a decision for surgery (suspicion of malignancy, symptoms, patient comorbidities) and cannot assume all surgical patients were presumed malignant. However, it is highly likely that those patients that did not go to surgery immediately following the initial draw were presumed to be benign by the physician, and this is supported by the high specificity in physician assessment alone [33-35]. It follows that the specificity of MIA3G measured for this population closely approximates the actual specificity in the absence of all surgical information, and that the resulting NPV is also representative.

In published data on MIA and MIA2G [36,38,39], the collection of an independent physician assessment permitted authors to demonstrate that the “OR” combination of biomarker and physician assessment yielded improved sensitivity for detecting malignancy. However, this reduced the specificity of the test. A similar algorithm for assessing potentially benign masses is not recommended, as it would tend to over-diagnose patients as at risk, but data from the retrospective studies point to more favorable outcomes when physician and biomarker assessments are combined. Physicians were generally good at assessing a benign mass, even in this population of patients where all enrolled patients were scheduled for surgery. For instance, Coleman et al. [36] showed in data from the OVA500 study—which contained specimens later used to either develop or validate MIA3G—that physician assessment alone had a specificity of 92.8% (95% CI: 89.8%-94.9%) for all evaluable subjects. Although the sensitivity in that study was 73.9% (95% CI: 64.1%-81.8%), the addition of MIA2G increased the sensitivity to 93.5%. In the stratified prospective MIAB dataset, MIA3G was able to identify 18/22 malignancies as an elevated risk that physicians assessed to be benign in the retrospective set, while it identified all the malignancies that physicians also assessed as malignant. It should be noted that MIA3G also identified 180 benign patients as indeterminate (false positive rate of 12.6%), and this suggests a that MIA3G might benefit from incorporation into clinical algorithms, or further neural network training against false positives to ameliorate these classification errors.

TABLE 16
Sensitivity, specificity, negative predictive value
(NPV) and positive predictive value (PPV) as a function
of MIAG3G score in data from the MIAB dataset.

| MIA3G Score | Sensitivity | Specificity | NPV | PPV |
|---|---|---|---|---|
| 0 | 100.0% | 0.0% | NA | 1.5% |
| 0.5 | 95.5% | 47.0% | 99.9% | 2.7% |
| 1 | 90.9% | 64.4% | 99.8% | 3.8% |
| 1.5 | 86.4% | 70.4% | 99.7% | 4.3% |
| 2 | 81.8% | 76.3% | 99.6% | 5.0% |
| 2.5 | 81.8% | 78.9% | 99.6% | 5.6% |
| 3 | 81.8% | 81.3% | 99.7% | 6.3% |
| 3.5 | 81.8% | 82.9% | 99.7% | 6.9% |
| 4 | 81.8% | 84.5% | 99.7% | 7.5% |
| 4.5 | 81.8% | 86.0% | 99.7% | 8.2% |
| 5 | 81.8% | 87.4% | 99.7% | 9.1% |
| 5.5 | 81.8% | 88.3% | 99.7% | 9.7% |
| 6 | 81.8% | 89.2% | 99.7% | 10.5% |
| 6.5 | 81.8% | 90.1% | 99.7% | 11.3% |
| 7 | 77.3% | 91.2% | 99.6% | 11.9% |
| 7.5 | 77.3% | 92.0% | 99.6% | 12.9% |
| 8 | 77.3% | 92.7% | 99.6% | 14.0% |
| 8.5 | 72.7% | 94.2% | 99.6% | 16.2% |
| 9 | 68.2% | 95.7% | 99.5% | 19.7% |
| 9.5 | 59.1% | 97.8% | 99.4% | 28.9% |
| 10 | 0.0% | 100.0% | 98.5% | NA |

The performance of MIA3G across the scores (Table 16) indicated the threshold MIA3G value of >5.0 for indeterminate results did not result in the highest performance for the PRW data set. Lower values of MIA3G in this set would result in higher sensitivity without much of a drop in specificity. The low prevalence may have been an influence on sensitivity. In the PRW data set with only 10 malignancies, the cutoff at 5.0 had an NPV of over 99%. At an MIA3G score of 2.5, the sensitivity would be 60%, and the specificity 79%, without impacting NPV. The performance of MIA3G across a range of cutoffs and prevalence, and the stability of NPV (Table 9) suggest that physicians may be able to interpret a risk level relevant to the clinical and pathological parameters of the patient or cohort they are addressing. A physician may choose a more conservative approach in patients with comorbidities and patients who may need to or want to delay surgery due to personal or professional reasons. In a higher prevalence population, the PPV will increase, and physicians may choose to use a cutoff that supports a higher PPV and specificity to identify patients at risk for ovarian malignancy. In such scenarios, as supported by these diverse data sets, the NPV provides confidence in the high probability of benign adnexal mass. This is also reflected in the probability of malignancy as a function of the MIA3G score. As FIGS. 9 and 12 (right) show, the probability of an abnormal (malignant?) mass as a function of the MIA3G score is not significantly different between the MIAB and PRW studies. From the logistic regression, the upper limit of the 95% CI of the probability of malignancy was 3.7% for the MIAB study and 6.4% for the PRW study at a threshold value of 5.0.

The results described in the aforementioned Examples were obtained using the following materials and methods.

### MIA3G Algorithm Development and Description

MIA3G is a proprietary deep feed-forward neural network (DNN)-based algorithm developed with the aim: low and elevated risk of malignancy. Data from a heterogenous set of 3,067 patient samples from previous clinical studies [38-40] was randomly assigned to training/testing and validation sets to derive and characterized the performance of the algorithm. All patients had undergone to surgery and thus had pathology confirmation of benign or malignant adnexal mass. The sample size of the malignant and benign cohorts was further balanced for algorithm training using a modification of the synthetic minority oversampling (SMOTE) [Example 1]. The following features: age, menopausal status, and seven protein biomarker measurements was trained via a neural network to known histopathological diagnoses of ovarian malignancy (malignant vs non-malignant) as the labels. Seven biomarkers used are cancer antigen 125 (CA125), human epididymis protein 4 (HE4), beta-2 microglobulin (B2M), apolipoprotein A-1 (ApoA1), transferrin (TRF), Prealbumin, (PreAlb), and follicle-stimulating hormone (FSH). MIA3G algorithm utilized multiple hidden layers each with their own weighted nodes and activation functions [41]. The neural network was regularized using node dropout to reduce overfitting where a percentage of the nodes are randomly omitted from each hidden layer during training [10]. The final layer of the neural network had two nodes and uses the softmax function to assign the probability of binary classification as low or elevated risk of malignancy. Further details of the classifier development have been previously described [Example 1].

The MIA3G test score was derived from the MIA3G algorithm. It was calculated as the softmax probability of elevated risk of malignancy scaled by 10, rounded down using a ‘floor’ function and binning into units of 0.5. The validated threshold value of a MIA3G softmax-high score of 0.5 (MIA3G score of 5.0) was used.

### Nomenclature

To delineate the differences across MIA3G test, surgical histology, and physicians' assessment outcomes the following terminology was used. In the retrospective studies where physicians were required to provide an independent assessment of the adnexal mass, the terms assessment benign and assessment malignant were used. The results of MIA3G were labeled as low risk of malignancy and indeterminate depending on whether the test result was above or below the score threshold, respectively. The diagnostic accuracy of physician assessment or MIA3G was evaluated against the “gold standard” of surgical histology which is referred to as histologically benign or histologically malignant.

### Data and Ethics

All data was obtained from adult patients who provided informed consent to participate in the research. All research was carried out under Institutional Review Board (IRB)-approved protocols. Protocol numbers are provided in Table 10.

### Studies and Sample Sets

This study presented validation of datasets—both retrospective and prospective—from multiple studies spanning multiple centers. Broadly, the inclusion criteria for these studies were as follows: 1) Patient age >18 years, 2) informed consent provided by the patient to participate in research, 3) patient agreeable to phlebotomy, 4) patient had a documented adnexal mass. The adnexal mass was confirmed by imaging (CT, TVUS or MRI) prior to enrollment. In the retrospective studies, all patients were scheduled for surgical intervention within 3 months of imaging. Exclusion criteria included a diagnosis of malignancy in the previous 5 years (except nonmelanoma skin cancers). Exclusion criteria also included adnexal surgery within six weeks prior to enrollment in the study.

Retrospective studies had previously been used to develop and validate MIA, MIA2G [38,39], and MIA3G [Example 1]. Because these data sets had information on physicians' independent clinical assessment of the malignancy of the mass, consistent with the intended uses of MIA and MIA2G, it was possible to stratify patients based on this assessment. Data from the assessment of benign patients comprised the “Multivariate Index Assay Benign” (MIAB) dataset.

The validation also included samples from ongoing prospective studies (Table 10), which was referred to as the “prospective real-world” (PRW) study. Data and sample collection protocols were identical for all samples. The subjects had a documented adnexal mass and were not yet scheduled for surgery. Patients were stratified on enrollment into cohorts A, B, or C based on physician determination. Cohort A comprised patients who had a mass and were symptomatic with symptoms such as pelvic pain, bloating or frequent urination and, as per physician's assessment, signs of potential malignancy on imaging, for example: complex cyst, solid mass, ascites. Cohort B comprised patients who were asymptomatic but discovered to have adnexal mass on exam or imaging. Cohort C consisted of those with known genetic risk or family history of ovarian cancer, and were permitted enrollment without an adnexal mass, although only patients with a documented adnexal mass from this cohort were included in this analysis.

For patients who did not immediately go to surgery within the period of this study, there may have been multiple blood draws to follow changes in biomarkers. Data from follow-up draws have not been included. Blood was drawn at the time of enrollment and batch-tested asynchronously for biomarkers and MIA3G test score determination. The physician was not provided MIA3G results at any point in the trial. At the physician's request, they could receive either CA125 results or MIA results to augment clinical decision-making.

A high-prevalence “independent assessment” (IA) set was assembled using a combination of 1) benign samples from the three prospective studies mentioned in Table 10, and 2) commercially-sourced serum samples from Accio Biobank Online (SHARE Bio-repository, Spectrum Health Network) and USBioLab (Fox Chase Cancer Center) These samples were obtained from patients with a documented adnexal mass which was planned for surgical intervention within 3 months of imaging (CT, TVUS or MRI)

### Determination of Serum Biomarker Values

The serum biomarker values for the prospective studies RP-08-2020, RP-09-2020, RP05-2019 were generated and run at a CAP-accredited CLIA laboratory (Aspira Labs, Austin TX). For patients in these protocols, a pre-operative blood sample of approximately 8.5 mL was collected into a serum processing tube and separated with centrifugation within 1-6 hours of collection. The sample was stored at 2-8 degrees C. and shipped to the laboratory on wet ice within 8 d of collection. All serum biomarker concentrations were determined on the Roche cobas 6000 clinical analyzer, utilizing the c501 and e601 modules and Roche Diagnostics' clinical assays. Biomarkers were run using assays that had passed rigorous lot acceptance criteria per laboratory QA/QC procedures. All measurements were performed on coded samples (blinded to patient demographics and/or pathology outcome).

### Statistics and Data Analysis

MIA3G scores for all patient cases were generated in the R Statistical Programming Language (ver 4.2.1) [42] using Tensorflow through the Keras interface (ver 2.4.0). The performance of MIA3G on the validation cohorts was also performed in R Statistical using the epiR library (ver 2.0.50) to generate estimates and confidence intervals of the binomial statistics. Confidence intervals were generated using Wilson's method [43]. PPV and NPV as a function of prevalence were calculated using the following formulae:

$$PPV = \frac{Sensitivity \times Prevalence}{Sensitivity \times Prevalence + ( 1 - Specificity \times ( 1 - Prevalence )}$$

$$NPV = \frac{Specificity \times ( 1 - Prevalence )}{( 1 - Sensitivity ) \times Prevalence + Specificity \times ( 1 - Prevalence )}$$

Bootstrapping approach employed to estimate performance as a function of prevalence was performed in R using the sample slice (replacement=TRUE) function of the dplyr library (ver 1.0.10). A total of 5,000 samples was generated to titrate prevalences from 1 to 10% malignancies. Principal component analysis was performed using prcomp from the base stats package of the R and visualized using the factoextra library (ver 1.0.7).

### Example 9: Serial Monitoring of Ovarian Cancer Risk in Patients with Adnexal Mass

Data was presented from ongoing multisite clinical studies in which total 924 patients presented with adnexal masses were enrolled. Follow-up visits were scheduled at clinician's discretion which may have included imaging and blood collection. Specimens were processed for serum and run on a clinical analyzer. MIA3G score from 0-10 was calculated using seven serum biomarkers coupling with patient age and menopausal status based on previously published neural network-based algorithm. MIA3G with NPV of 99.7% (CI:99.2-99.9) was used to risk stratify the patient with an adnexal mass into low probability of malignancy or indeterminate with a validated cut off at 5.0. For this analysis, MIA3G scores were also binned into the following Zones: 1(0-2.49), 11(2.50-4.99) and 11(5.00-10.00).

Of 924 enrolled patients, 538 patients had completed clinical and biomarker data on initial study draw. Of these, 145 had at least one follow-up test and 31 had at least two follow-up test. Median duration to first follow-up test was 108 d (˜3.6 mon), and median duration to second follow-up test was 272 d (˜9.1 mon). Follow-up in 3 MIA3G score zones at the 0-3.6 and 3.6-9.1 mon interval consistently showed 88% patients within zone I remained unchanged, whereas 12% of patients moved to zones II and III. In the 0-3.6 mon interval, the median change in MIA3G score from I to II was 2.25 (n=9, range 0.54-3.52) and from I to III was 5.92 (n=8, range 5.08-7.62). In the 3.6-9.1 mon testing interval, the median change in MIA3G score from zone I to II was 3.11 (n=2, range 2.75 to 3.46) and the single patient change from I to III was 6.89. Across both testing intervals, 17%-50% of patients in zone II remained unchanged while approximately 25% patients had score increase to zone III. Importantly, 50% of patients in zone III remained unchanged in the absence of clinical intervention whereas 50% had MIA3G score reduction in association with clinical management.

These results indicate that the serial follow-up with MIA3G is recommended to monitor the clinical status of the adnexal mass every 3 months, MIA3G score changes of >2.25 suggest a clinical implication indicative of clinical follow-up, and finally the MIA3G is a suitable tool for the effectiveness of clinical management of adnexal mass.

### REFERENCES

1. Ueland F R, Fredericks T I. Ovarian masses: Surgery or surveillance?OBG Manag. 2018; 30(6):17-24, 26.
2. Rim S H, Hirsch S, Thomas C C, Brewster W R, Cooney D, Thompson T D, Stewart S L. Gynecologic oncologists involvement on ovarian cancer standard of care receipt and survival. World J Obstet Gynecol. 2016; 5(2):187-196. doi: 10.5317/wjog.v5.i2.187. Epub 2016 May 10.
3. Cancer of the Ovary—Cancer Stat Facts. SEER: Surveillance, Epidemiology, and End Results Program. Published 2021. Accessed Nov. 11, 2021. https://seer.cancer.gov/statfacts/html/ovary.html
4. May T, Oza A. Conservative management of adnexal masses. Lancet Oncol. 2019; 20(3):326-327. doi:10.1016/S1470-2045(18)30939-2
5. Doubeni C A, Doubeni A R, Myers A E. Diagnosis and Management of Ovarian Cancer. Am Fam Physician. 2016; 93(11):937-944.
6. Badgwell D, Bast R C Jr. Early detection of ovarian cancer. Dis Markers. 2007; 23(5-6):397-410. doi:10.1155/2007/309382
7. Choi J H, Sohn G S, Chay D B, Cho H B, Kim J H. Preoperative serum levels of cancer antigen 125 and carcinoembryonic antigen ratio can improve differentiation between mucinous ovarian carcinoma and other epithelial ovarian carcinomas. Obstet Gynecol Sci. 2018; 61(3):344-351. doi:10.5468/ogs.2018.61.3.344
8. Kommoss F, Lehr H A. Keimstrang-Stromatumoren des Ovars: Aktuelle Aspekte, insbesondere zu Granulosazelltumoren, Sertoli-Leydig-Zell-Tumoren und Gynandroblastomen [Sex cord-stromal tumors of the ovary: Current aspects with a focus on granulosa cell tumors, Sertoli-Leydig cell tumors, and gynandroblastomas]. Pathologe. 2019; 40(1):61-72. doi:10.1007/s00292-018-0562-3
9. Artificial Intelligence. National Cancer Institute. Published Aug. 19, 2020. Accessed Nov. 15, 2021. https://www.cancer.gov/research/areas/diagnosis/artificial-intelligence
10. Srivastava N, Hinton G, Krizhevsky A, Salakhutdinov R. Dropout: A Simple Way to Prevent Neural Networks from Overfitting. Journal of Machine Learning Research. 2014; 15(1):1929-1958. doi:https://dl.acm.org/doi/10.5555/2627435.2670313
11. Bast R C Jr, Klug T L, St John E, et al. A radioimmunoassay using a monoclonal antibody to monitor the course of epithelial ovarian cancer. N Engl J Med. 1983; 309(15):883-887. doi:10.1056/NEJM198310133091503
12. Drapkin R, von Horsten H H, Lin Y, et al. Human epididymis protein 4 (HE4) is a secreted glycoprotein that is overexpressed by serous and endometrioid ovarian carcinomas. Cancer Res. 2005; 65:2162-9.
13. Zhang Z, Bast R C Jr, Yu Y, et al. Three biomarkers identified from serum proteomic analysis for the detection of early stage ovarian cancer. Cancer Res. 2004; 64(16):5882-5890. Doi:10.1158/0008-5472.CAN-04-0746
14. Yang H S, Li Y, Deng H X, Peng F. Identification of beta2-microglobulin as a potential target for ovarian cancer. Cancer Biol Ther. 2009; 8(24):2323-2328. Doi:10.4161/cbt.8.24.9982
15. Kozak K R, Su F, Whitelegge J P, Faull K, Reddy S, Farias-Eisner R. Characterization of serum biomarkers for detection of early stage ovarian cancer. Proteomics. 2005; 5(17):4589-4596. doi:10.1002/pmic.200500093
16. Zhang Z. An In Vitro Diagnostic Multivariate Index Assay (IVDMIA) for Ovarian Cancer: Harvesting the Power of Multiple Biomarkers. Rev Obstet Gynecol. 2012; 5(1):35-41.
17. Clinicaltrials.gov. A Multivariate Index Assay for Ovarian Cancer Risk Assessment in Women With Adnexal Mass and High-Risk Germline Variants. clinicaltrials.gov. Published Aug. 23, 2021. Accessed Aug. 30, 2021. https://clinicaltrials.gov/ct2/show/NCT04487405
18. Zhang Z, Bullock R G, Fritsche H. Adnexal mass risk assessment: a multivariate index assay for malignancy risk stratification. Future Oncology. 2019; 15(33):3783-3795. doi:10.2217/fon-2019-0479
19. Bristow R E, Smith A, Zhang Z, et al. Ovarian malignancy risk stratification of the adnexal mass using a multivariate index assay. Gynecol Oncol. 2013; 128(2):252-259. doi:10.1016/j.ygyno.2012.11.022
20. Urban R R, Smith A, Agnew K, Bonato V, Goff B A. Evaluation of a Validated Biomarker Test in Combination With a Symptom Index to Predict Ovarian Malignancy. Int J Gynecol Cancer. 2017; 27(2):233-238. doi: 10.1097/IGC.0000000000000873
21. Clinicaltrials.gov. Whole Blood Collection Protocol For Ovarian Assay Clinical Trial In Women With Ovarian Tumors. clinicaltrials.gov. Published Apr. 25, 2008. Accessed Aug. 30, 2021. https://clinicaltrials.gov/ct2/show/NCT00436189
22. Open Clinical Trials: Other. Spectrum Health. Published July 2019. Accessed Aug. 30, 2021. https://www.spectrumhealth.org/-/media/spectrumhealth/documents/clinical-research/available-clinical-trials-pdfs/other-health-clinical-trials/other-open-clinical-trials.pdf
23. Han H, Wang W Y, Mao B H, et al. Borderline-SMOTE: A New Over-Sampling Method in Imbalanced Data Sets Learning. Advances in Intelligent Computing. ICIC 2005.

Lecture Notes in Computer Science, vol 3644. Springer, Berlin, Heidelberg. https://doi.org/10.1007/11538059_91
24. Kuhn M, Wing J, Weston S, et al. Package “Caret.”; 2021. Accessed Aug. 30, 2021. https://cran.r-project.org/web/packages/caret/caret.pdf
25. Berek J S, Bast R C Jr. Nonepithelial Ovarian Cancer. In: Kufe D W, Pollock R E, Weichselbaum R R, et al., editors. Holland-Frei Cancer Medicine. 6th edition. Hamilton (ON): B C Decker; 2003. Available from: www.ncbi.nlm.nih.pov/books/NBK13342/
26. Dropout: A Simple Way to Prevent Neural Networks from Overfitting Nitish Srivastava, Geoffrey Hinton, Alex Krizhevsky, Ilya Sutskever, Ruslan Salakhutdinov; 15(56):1929-1958, 2014.
27. Han, H., Wang, W. Y. and Mao, B. H. Borderline-SMOTE: a new over-sampling method in imbalanced data sets learning. In Proceedings of the 2005 international conference on Advances in Intelligent Computing—Volume Part I (ICIC'05), De-Shuang Huang, Xiao-Ping Zhang, and Guang-Bin Huang (Eds.), Vol. Part I. Springer-Verlag, Berlin, Heidelberg, 2005. 878-887. DOI=http://dx.doi.org/10.1007/11538059_91
28. Tholen D W, Kallner A, Kennedy J W, et al. EP05-A2: Evaluation Of Precision Performance Of Quantitative Measurement Methods; Approved Guideline—Second Edition. Clinical and Laboratory Standards Institute. 2014:1-39
29. Greenlee R, Kessel B, Williams C, Riley T, Ragard L, Hartge P, et al. Prevalence, incidence, and natural history of simple ovarian cysts among women >55 years old in a large cancer screening trial. Am J Obstet Gynecol. (2010)
30. McDonald J, Modesitt S. The incidental postmenopausal adnexal mass. Clin Obstet Gynecol. (2006) 49:506-16
31. Smith-Bindman R, Poder L, Johnson E, Miglioretti D. Risk of malignant ovarian cancer based on ultrasonography findings in a large unselected population. JAMA Internal Med. (2019) 179:71-7
32. Timmerman D, Ameye L, Fischerova D, Epstein E, Melis G, Guerriero S, et al. Simple ultrasound rules to distinguish between benign and malignant adnexal masses before surgery: prospective validation by IOTA group. Multicenter Stud. (2010) 341:c6839.
33. Sadowski E, Paroder V, Patel-Lippmann K, Robbins J, Barroilhet L, Maddox E, et al. Indeterminate adnexal cysts at U S: prevalence and characteristics of ovarian cancer. Radiology. (2018) 287:1041-9.
34. Pavlik E, Ueland F, Miller R, Ubellacker J, DeSimone C, Elder J, et al. Frequency and disposition of ovarian abnormalities followed with serial transvaginal ultrasonography. Obstet Gynecol. (2013) 122:210-7.
35. Froyman W, Landolfo C, De Cock B, Wynants L, Sladke'cius P, Testa A, et al. Risk of complications in patients with conservatively managed ovarian tumours (IOTA5): a 2-year interim analysis of a multicentre, prospective, cohort study. Lancet Oncol. (2019) 20:448-58.
36. Coleman R, Herzog T, Chan D, Munroe D, Pappas T, Smith A, et al. Validation of a second-generation multivariate index assay for malignancy risk of adnexal masses. Am J Obstet Gynecol. (2016) 215:el-11.
37. Zhang Z, Chan D. The road from discovery to clinical diagnostics: lessons learned from the first FDA-cleared in vitro diagnostic multivariate index assay of proteomic biomarkers. Cancer Epidemiol Biomarkers Prev. (2010) 19:2995-9.
38. Ueland F, Desimone C, Seamon L, Miller R, Goodrich S, Podzielinski I, et al. Effectiveness of a multivariate index assay in the preoperative assessment of ovarian tumors. Obstet Gynecol. (2011) 117:1289-97.
39. Bristow R, Hodeib M, Smith A, Chan D, Zhang Z, Fung E, et al. Impact of a multivariate index assay on referral patterns for surgical management of an adnexal mass. Am J Obstet Gynecol. (2013) 209:581.el-8.
40. Urban R, Pappas T, Bullock R, Munroe D, Bonato V, Agnew K, et al. Combined symptom index and second-generation multivariate biomarker test for prediction of ovarian cancer in patients with an adnexal mass. Gynecol Oncol. (2018) 150:318-23.
41. Kukaeka J, Golkov V, Cremers D. Regularization for deep learning: a taxonomy. arXiv. (2017): preprint arXiv:171010686. doi: 10.48550/arXiv.1710.10686
42. R Core Team. R: A language and environment for statistical computing. Vienna: R Foundation for Statistical Computing (2022). Available online at: www.R-project. org/
43. Wilson E. Probable inference, the law of succession, and statistical inference. Journal Am Stat Associat. (1927) 22:209-12.
44. Lok I, Sahota D, Rogers M, Yuen P. Complications of laparoscopic surgery for benign ovarian cysts. J Am Assoc Gynecol Laparosc. (2000) 7:529-34.
45. Yoong W, Fadel M, Walker S, Williams S, Subba B. Retrospective cohort study to assess outcomes, cost-effectiveness, and patient satisfaction in primary vaginal ovarian cystectomy versus the laparoscopic approach. J Minim Invas Gynecol. (2016) 23:252-6.
46. Moore R, Miller M, Disilvestro P, Landrum L, Gajewski W, Ball J, et al. Evaluation of the diagnostic accuracy of the risk of ovarian malignancy algorithm in women with a pelvic mass. Obstet Gynecol. (2011) 118:280-8.
47. Mohaghegh P, Rockall A. Imaging strategy for early ovarian cancer: characterization of adnexal masses with conventional and advanced imaging techniques. Radio Graphics. (2012) 32:1751-73.
48. Schumer S, Cannistra S. Granulosa cell tumor of the ovary. J Clin Oncol. (2003) 21:1180-9.
49. Lantzsch T, Stoerer S, Lawrenz K, Buchmann J, Strauss H, Koelbl H. Sertoli-leydig cell tumor. Arch Gynecol Obstet. (2001) 264:206-8.
50. Menon U, Gentry-Maharaj A, Burnell M, Singh N, Ryan A, Karpinskyj C, et al. Ovarian cancer population screening and mortality after long-term follow-up in the U K collaborative trial of ovarian cancer screening (UKCTOCS): a randomised controlled trial. Lancet. (2021) 397:2182-93.
51. Carugno J, Naem A, Ibrahim C, Ehinger N, Moore J, Garzon S, et al. Is color doppler ultrasonography reliable in diagnosing adnexal torsion?A large cohort analysis. Minim Invasive Ther Allied Technol. (2022) 31:620-7

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents, publications, and accession numbers mentioned in this specification, are herein incorporated by reference to the same extent as if each independent patent, publication, and accession number was specifically and individually indicated to be incorporated by reference.

What is claimed is:

1. A computer implemented method for assessing a subject's risk of having ovarian cancer, the method comprising:
a) receiving, by one or more computing devices each comprising a processor and a memory, a plurality of signals, each signal representing a value of a biomarker from a panel of biomarkers detected in a biological sample derived from a subject having an adnexal mass, wherein the panel of biomarkers comprises Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH);
b) receiving, by the one or more computing devices, an age value representing the age of the subject and a menopausal value representing the menopausal state of the subject; and
c) determining, using an artificial neural network stored in the one or more computing devices, a score based on the plurality of signals, the age value, and the menopausal value, wherein the score represents whether the adnexal mass is benign, or the adnexal mass has an indeterminate risk of malignancy.

2. The method of claim 1, wherein the plurality of signals each represent a biomarker spectrum peak detected for each biomarker of the panel of biomarkers.

3. The method of claim 1, wherein the artificial neural network is a deep feed-forward neural network.

4. The method of claim 3, wherein the artificial neural network comprises a plurality of input nodes, a plurality of hidden nodes, and a plurality of output nodes.

5. The method of claim 4, wherein each of the input nodes comprises a memory location for storing an input value, each input value corresponding to a different value from one of the plurality of signals, the age value, or the menopausal value.

6. The method of claim 4, wherein the plurality of hidden nodes is organized into a plurality of hidden layers, each hidden layer having a different set of weighted nodes and/or activation functions.

7. The method of claim 4, wherein the plurality of output nodes comprises a first output node and a second output node, the first output node including a memory location for storing a first output value indicating the probability of a first classification, and the second output node including a memory location for storing a second output value indicating the probability of a second classification, wherein the first classification represents a benign adnexal mass and the second classification represents an adnexal mass having an indeterminate risk of malignancy.

8. The method of claim 4, wherein the artificial neural network uses the softmax function to assign the first and second output values.

9. The method of claim 4, wherein the artificial neural network is regularized using node dropout to reduce overfitting.

10. The method of claim 4, wherein the artificial neural network is trained using supervised training.

11. The method of claim 4, wherein the artificial neural network is trained using a training set comprising a set of malignant samples and a set of benign samples.

12. The method of claim 11, wherein the number of samples in the set of malignant samples and the number of samples in the set of benign samples is balanced using a synthetic minority oversampling technique (SMOTE) to create a balanced training set.

13. The method of claim 12, wherein the SMOTE comprises balancing minority and majority classes within the training set by creating synthetic samples near the decision boundary.

14. The method of claim 12, wherein the balanced training set has an equal amount of malignant samples and benign samples.

15. The method of claim 12, wherein the training set has 100-500 malignant samples in the set of malignant samples.

16. The method of claim 14, wherein the artificial neural network is trained by attaching a higher weight to detection of malignant samples.

17. The method of claim 16, wherein the imaging is transvaginal ultrasonography (TVUS).

18. The method of claim 17, wherein the characterization of the adnexal mass as non-malignant or asymptomatic comprises using TVUS imaging over the course of at least 5 months without an increase in adnexal mass size.

19. A method for training an artificial neural network for detecting the risk of ovarian cancer in a subject, the method comprising:
a) collecting a training set comprising a set of malignant adnexal mass samples and a set of benign adnexal mass samples;
b) balancing the number of samples in each of the set of malignant adnexal mass samples and the set of benign adnexal mass samples by synthetically creating samples near the decision boundary; and
c) training the artificial neural network on the training set, wherein the training comprises regularizing the artificial neural network using node dropout and attaching a higher weight to identifying malignant samples.

20. A method for monitoring a subject's risk of having ovarian cancer, comprising:
(a) assessing the subject at a first time point in a plurality of time points using the method of claim 1; and
(b) repeating step (a) in one or more biological samples from the subject identified as having an intermediate or low ovarian cancer risk, or as having a benign adnexal mass, at one or more following time points in the plurality of time points, thereby monitoring the subject.

21. A method of conservative management of an adnexal mass in a selected subject, the method comprising:
(a) selecting a subject having an adnexal mass and at least one contraindication to surgical intervention;
(b) characterizing a panel of markers in a biological sample derived from the selected subject using a computer-implemented method to determine a score, wherein the markers in the panel of markers comprise cancer antigen 125 (CA125), human epididymis protein 4 (HE4), beta-2 microglobulin (B2M), apolipoprotein A-1 (ApoA1), transferrin, transthyretin, and follicle stimulating hormone (FSH), and wherein the score identifies the subject as having a benign adnexal mass, or having an adnexal mass having an indeterminate risk of malignancy; and
(c) conservatively managing the adnexal mass when the score identifies the subject as having a benign adnexal mass.
